SEQUENCE LISTING (10 sequences)

SEQ ID NO: 1 - protein, 16 aa, Artificial Sequence
  Synthetic Peptide
AGFSGPLGMWSAGSFG

SEQ ID NO: 2 - DNA, 22 bp, Artificial Sequence
  BRAF-specific forward oligonucleotide primer
gcatctcacctcatcctaacac

SEQ ID NO: 3 - DNA, 22 bp, Artificial Sequence
  BRAF-specific reverse oligonucleotide primer
ctagtaactcagcagcatctca

SEQ ID NO: 4 - DNA, 22 bp, Artificial Sequence
  BRAF-specific sequencing primer
gcatctcacctcatcctaacac

SEQ ID NO: 5 - RNA, 19 nt, Artificial Sequence
  BRAF-specific siRNA
gcuacagagaaaucucgau

SEQ ID NO: 6 - RNA, 21 nt, Artificial Sequence
  BRAF-specific siRNA
aacagucuacaagggaaagug

SEQ ID NO: 7 - RNA, 19 nt, Artificial Sequence
  BRAF-specific siRNA
gcuacagagaaaucucgau

SEQ ID NO: 8 - DNA, 46 bp, Homo sapiens
aaaaataggtgattttggtctagctacagagaaatctcgatggagt

SEQ ID NO: 9 - DNA, 46 bp, Homo sapiens; identical to SEQ ID NO: 8

SEQ ID NO: 10 - DNA, 45 bp, Artificial Sequence
  consensus sequence
aaaaataggtgattttggtctagctacaggaaatctcgatggagt

(19) United States
(12) Patent Application Publication
(10) Pub. No.: US 2017/0056327 A1
(43) Pub. Date: Mar. 2, 2017

(54) MICRO/NANO COMPOSITE DRUG DELIVERY FORMULATIONS AND USES THEREOF

(71) Applicant: The Methodist Hospital, Houston, TX (US)

(72) Inventors: Yu MI, Houston, TX (US); Mauro FERRARI, Houston, TX (US)

(21) Appl. No.: 15/247,753

(22) Filed: Aug. 25, 2016

Related U.S. Application Data
(60) Provisional application No. 62/209,598, filed on Aug. 25, 2015.

(51) Int. Cl. A61K 9/16; A61K 31/713; A61K 31/337; A61K 9/127

(52) U.S. Cl. CPC A61K 9/1647; A61K 9/127; A61K 9/1641; A61K 9/1611; A61K 31/713; A61K 31/337

(57) ABSTRACT

Disclosed are micro/nano composite drug delivery compositions for use in diagnosis, prophylaxis, treatment and/or amelioration of one or more symptoms of a mammalian disease, disorder, dysfunction, or abnormal condition. In illustrative embodiments, pharmaceutical formulations comprising these composites are provided that are useful in methods for targeting selected mammalian cells and tissues, particularly human lung tissue, and delivering one or more therapeutic agents, particularly in the treatment of human lung cancers, such as melanoma lung metastases.

### CROSS-REFERENCE TO RELATED APPLICATIONS

The present application claims priority to U.S. Provisional Patent Application No. 62/209,598, filed Aug. 25, 2015 (pending; Atty. Dkt. No. 37182.195PV01); the contents of which is specifically incorporated herein in its entirety by express reference thereto.

### STATEMENT REGARDING FEDERALLY SPONSORED RESEARCH OR DEVELOPMENT

This invention was made with government support under Grant Nos. W81XWH-09-1-0212 and W81XWH-12-1-0414, awarded by the United States Department of Defense, and Grant Nos. U54CA143837 and U54CA151668, awarded by the National Institutes of Health. The government has certain rights in the invention.

### NAMES OF THE PARTIES TO A JOINT RESEARCH AGREEMENT

Not Applicable.

### BACKGROUND OF THE INVENTION

### Field of the Invention

The present invention generally relates to the fields of medicine and oncology. In particular, the invention provides improved chemotherapeutic compositions for the treatment and/or amelioration of one or more symptoms of human cancers. In particular, micro/nano composite dual vectors are provided, which are useful for targeting mammalian lung tissue, and for treating lung cancer, including metastases to the lung such as melanoma metastasis to the lung.

### Description of Related Art

Nanotechnology

Nanotechnology pertains to synthetic, engineerable objects that are nanoscale in dimensions or have critical functioning nanoscale components, leading to novel, unique properties (Ferrari, 2005; Theis et al., 2006). These emergent characteristics arise from the material's large surface area and nanoscopic size (Riehemann, 2009). Nanotechnology now occupies a niche as a burgeoning and revolutionary field within medicine known as nanomedicine, particularly within the field of oncology (Ferrari, 2005). One of the potential benefits of nanomedicine is the creation of nanoparticle-based vectors that deliver therapeutic cargo in sufficient quantity to a target lesion to enable a selective effect. This is a daunting task for all drug molecules, owing to the highly organized array of ‘biological barriers’ that the molecules encounter (Riehemann, 2009; Jain, 1989; Jain, 1999; Sakamoto et al., 2007).

The human body presents a robust defense system that is extremely effective in preventing injected chemicals, biomolecules, nanoparticles, and any other foreign agents from reaching their intended destinations. Biobarriers are sequential in nature, and therefore, the probability of reaching the therapeutic objective is the product of individual probabilities of overcoming each barrier (Ferrari, 2005; Ferrari, 2009). Sequentially (with respect to intravascular injections) these comprise: 1) enzymatic degradation; 2) sequestration by phagocytes of the reticulo-endothelial system (RES) (Caliceti and Veronese, 2003; Moghimi and Davis, 1994); 3) vascular endothelia (Mehta and Malik, 2006); 4) adverse oncotic and interstitial pressures in the tumor (Stohrer et al., 2000; Less et al., 1992); 5) cellular membranes, or subcellular organelles such as the nucleus and endosomes (Torchilin, 2006; Majumdar and Mita, 2006); and 6) molecular efflux pumps (Undevia et al., 2005). Without an effective strategy to negotiate these barriers, new or current therapeutic agents based on enhanced biomolecular selectivity may yield sub-optimal utility, simply because they reach the intended targets in very small fractions, with only 1 in 10,000 to 1 in 100,000 molecules reaching their intended site of action (Ferrari, 2005). Due to this narrow therapeutic window, marginal tolerability and considerable mortality often ensue (Canal et al., 1998).

Transport through different cellular compartments, and across one or more biological barriers/membranes, however, can be enhanced by optimization of particle size, shape, density, and surface chemistry of nanoparticle delivery vehicles. These parameters dominate transport of nanoparticle formulations administered intravenously, and regulate transport, margination, cell adhesion, selective cellular uptake, and sub-cellular trafficking of the particles within mammalian cells.

An early obstacle for intravascularly-administered therapeutics is the endothelial wall that forms the boundary between the circulatory system and tissue specific microenvironments. Specific adherence of delivery vectors to diseased vasculature provides a key to conquering this early barrier, as does the hijacking of cells bound for the inflammatory microenvironment of the lesion.

### Deficiencies in the Prior Art

In particular, what is lacking in the prior art are compositions and methods for efficiently overcoming various bio-barriers that employ multiple levels of targeting, spatial release of secondary carriers or therapeutics, simultaneous delivery of independent systems and/or systems capable of synergistic impact.

### BRIEF SUMMARY OF THE INVENTION

The present invention overcomes these and other inherent limitations in the art by providing, in a general sense, micro- and nano-composites that achieve combinational therapy of at least a first small interfering RNA (siRNA) in combination with at least a first chemotherapeutic drug for treatment of a first cancer, including, without limitation, mammalian lung cancers, such as human melanoma lung metastases. In particular embodiments, these micro- and nano-composite compositions have been shown to improve the accumulation of conventional chemotherapeutic-loaded nanoparticles in certain tissues, and in particular, in the lung, thus achieving better therapeutic effect within human lung tissues.

In particular embodiments, the micro- and nano-composites of the present disclosure have been employed to provide combinational therapy of siRNAs and chemotherapeutic drugs to treat human melanoma lung metastasis. These drug delivery compositions improve the accumulation of conventional therapeutics-loaded nanoparticles in the lung, and achieve better therapeutic effect over currently-available therapies.

The present disclosure provides in one aspect, poly(lactide-co-glycolide) (PLGA)-polyethylene glycol (PEG) (PLGA-PEG) nanoparticles conjugated to the surface of mesoporous silicon microdisks. Nanoparticles show rapid clearance and short retention time in lungs. The composite can help to transport nanoparticles to the lungs and accumulate the particles in lungs for better therapeutic effect. The strategy of conjugation eliminates concern for controlling pore size, and improves stability and loading doses in the composite.

Alternatively, nanoparticles may be manufactured out of any suitable polymeric material and/or combinations thereof, including for example, and without limitation, PCL-PEG, PLA-PEG, polyurethane (PU), and the like, or alternatively, from one or more inorganic materials, such as gold, or other metals, and oxides, such as iron oxide and combinations thereof. Similarly, nanoparticles may include micelles, liposomes, lipid particles, or may even include prodrugs or other compounds that are directly loaded into the pores of the silicon microparticles without being pre-incorporated into one or more distinct nanoparticles themselves.

The nanoparticle component(s) of the present drug delivery system are preferably fabricated in a suitable dimension for incorporation into the selected microparticles. The inventors contemplate that nanoparticles having an average size of between about 60 nm and about 500 nm are particularly preferred in the practice of the invention.

In an illustrative embodiment, a silicon microdisk (having a size of 2.6 μm in diameter and 0.7 μm in height, was fabricated, which contained a plurality of 60 nm-diameter pores. Next, siRNA-loaded liposomes were prepared and loaded into the pores of the silicon microparticles. Finally, chemotherapeutic agent (in an illustrative example, docetaxel)-loaded PLGA-PEG nanoparticles were conjugated to the surface of the silicon microparticles.

The microparticle component(s) of the disclosed drug delivery systems are preferably fabricated in a suitable dimension to permit sufficient incorporation of the selected nanoparticles, while also facilitating efficient delivery of the active agent(s) comprised within such nanoparticles. The inventors contemplate that microparticles having dimensions in the following ranges to be particularly preferred in the practice of the invention: microparticle average diameter:—from about 1 μm to about 10 μm; microparticle average height—from about 0.1 μm to about 5 μm; and microparticle average pore diameters—from 0 to about 60 nm.

The resulting micro/nano composite composition was administered systemically to an animal. In the lungs, the conjugated PLGA-PEG NPs on the surface of the microdisks, and the siRNA-loaded liposomes contained inside the pores were released through biodegradation of the silicon microdisks, and through enzyme-facilitated degradation of the MMP2/MMP9 substrate. The siRNA-loaded liposomes were then transported into the cancer cells by endocytosis and EPR effect. From the resulting micro/nano composite, the docetaxel in the PLGA-PEG NPs and the siRNA in the liposomes could be simultaneously released in the lung tissue, thereby achieving a synergistic, anti-tumoral effect.

In addition to siRNAs, small molecules including, without limitation, other nucleic acids (e.g., DNAs, tRNAs, mRNAs, ssDNAs, etc.), peptides, inhibitors (e.g., vemurafenib, trametinib, and the like), prodrugs, drugs, diagnostic markers, or any combinations thereof, may also be loaded into the nanoparticles and delivered to cells or tissues using the systems described herein.

The inventors have shown that the composite improve the lung accumulation retention of PLGA-PEG nanoparticles or liposomes, so that the accumulation of a chemotherapeutic (such as docetaxel) and one or more siRNAs is relatively increased. The morphology, controlled release, and biodegradation of the composites described herein have been tested in vitro. The cellular uptake efficiency, gene knockdown efficiency, and synergistic anti-cancer effect of the composite have been assessed in A375 human melanoma cells in vitro. The biodistribution and therapeutic effect of the composite are assessed on the A375 human melanoma lung metastasis mouse model.

The present disclosure provides compositions that improve the lung accumulation of conventional nanoparticles, and therefore improve the therapeutic effect of lung cancer or cancer lung metastasis. PLGA-PEG nanoparticles show many advantages in delivering hydrophobic therapeutics. However, they show short retention time in lungs and are less efficient for therapy of lung cancer or cancer lung metastasis.

The resulting silicon microparticles have been used to load pro-drugs, proteins, or small nanoparticles (such as micelles) inside the pores for cancer therapy. However, when one considers polymeric nanoparticles, which often show rigid structure and bigger size then the pores of a silicon microdisk, they cannot be loaded into the pores. The pore size of the porous silicon micro disk is often less than 60 nm for the requirement of stability.

In the present disclosure, polymeric nanoparticles were conjugated to the surface of silica microdisks. The resulting hybrid micro/nano-conjugate particles showed a high degree of stability and offered high loading amounts of nanoparticles in targeted tissues. Through chemical conjugation, the polymeric nanoparticles with size more than 100 nm can be transported by silicon microdisks. At the same time, small nanoparticles, such as liposomes and micelles, can also be loaded inside the pores prior to conjugation. Therefore, both the pores and surface of the silicon microdisks can be fully used. Drugs and/or siRNA can be delivered within the same composite, and co-localized delivery to the lungs can be achieved resulting in a synergistic, anti-cancer effect.

The disclosed micro/nano composite combines the advantages of both microdisks and nanoparticles as agents for drug delivery to selected tissues. Nanoparticles around 100 nm to 200 nm, such as polymeric nanoparticles and liposomes, show advantages in cellular uptake and EPR effect. Moreover, liposomes show good biocompatibility and high loading efficiency for hydrophilic agents such as siRNA. PLGA-PEG NPs show good stability and high loading efficiency for hydrophobic agents. However, they show rapid clearance by MPS in liver or spleen, as well as short retention time in lungs. Microdisks having approximately 2.6 μm diameters, and of approximately 0.7 μm in height showed excellent transport properties in mammalian blood. More importantly, they improve the lung accumulation by remain in the abundant and small capillary of lungs. By loading the liposomes inside the cores and conjugating the PLGA NPs on the surface of the microdisks, the biodistribution of the nanoparticles can be controlled, and they can be transported to the lungs. The prolonged retention time in lung tissues helps deliver more of the therapeutic molecules to the lesions within the lung tissue. The current design also facilitates combinational therapy by transporting both chemotherapeutic agents and siRNA at the same time in the same particles. The increased accumulation and synergistic effect of combination therapy improves upon current treatment modalities for lung cancer and lung cancer metastases.

Chemotherapeutic Methods and Use

Another important aspect of the present disclosure concerns methods for using the disclosed micro/nano composite drug delivery formulations for treating or ameliorating the symptoms of one or more forms of cancer, including, for example, a metastatic cancer, such as melanoma metastasis to the mammalian lung. Such methods generally involve administering to a mammal (and in particular, to a human in need thereof), one or more of the disclosed anticancer compositions, in an amount and for a time sufficient to treat (or, alternatively ameliorate one or more symptoms of) the lung cancer in an affected mammal.

In certain embodiments, the micro/nano composite drug delivery formulations described herein may be provided to the animal in a single treatment modality (either as a single administration, or alternatively, in multiple administrations over a period of from several hours (hrs) to several days (or even several weeks or several months) as needed to treat the particular cancer. Alternatively, in some embodiments, it may be desirable to continue the treatment, or to include it in combination with one or more additional modes of therapy, for a period of several months or longer. In other embodiments, it may be desirable to provide the therapy in combination with one or more existing, or conventional treatment regimens.

The present disclosure also provides for the use of one or more of the disclosed micro/nano composite drug delivery compositions in the manufacture of a medicament for therapy and/or for the amelioration of one or more symptoms of cancer, and particularly for use in the manufacture of a medicament for treating and/or ameliorating one or more symptoms of a mammalian cancer, such as human lung cancer.

The present invention also provides for the use of one or more of the disclosed micro/nano composite drug delivery formulations in the manufacture of a medicament for the treatment of cancer, and in particular, the treatment of human, metastatic melanoma cancer of the lung.

Therapeutic Kits

Therapeutic kits including one or more of the disclosed micro/nano composite drug delivery formulations and instructions for using the kit in a particular cancer treatment modality also represent preferred aspects of the present disclosure. These kits may further optionally include one or more additional anti-cancer compounds, one or more diagnostic reagents, one or more additional therapeutic compounds, or any combination thereof.

The kits of the invention may be packaged for commercial distribution, and may further optionally include one or more delivery devices adapted to deliver the micro/nano composite drug delivery composition(s) to an animal (e.g., syringes, injectables, and the like). Such kits typically include at least one vial, test tube, flask, bottle, syringe, or other container, into which the micro/nano composite drug delivery composition(s) may be placed, and preferably suitably aliquotted. Where a second pharmaceutical is also provided, the kit may also contain a second distinct container into which this second composition may be placed. Alternatively, the plurality of micro/nano composite drug delivery compositions disclosed herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Pharmaceutical Formulations

In certain embodiments, the present invention concerns formulation of one or more chemotherapeutic and/or diagnostic compounds in a pharmaceutically acceptable formulation of the micro/nano composite drug delivery formulations disclosed herein for administration to one or more cells or tissues of an animal, either alone, or in combination with one or more other modalities of diagnosis, prophylaxis, and/or therapy. The formulation of pharmaceutically acceptable excipients and carrier solutions is well known to those of ordinary skill in the art, as is the development of suitable dosing and treatment regimens for using the particular compositions described herein in a variety of treatment regimens.

In certain circumstances it will be desirable to deliver the disclosed chemotherapeutic compositions in suitably-formulated pharmaceutical vehicles by one or more standard delivery devices, including, without limitation, subcutaneously, parenterally, intravenously, intramuscularly, intrathecally, orally, intraperitoneally, transdermally, topically, by oral or nasal inhalation, or by direct injection to one or more cells, tissues, or organs within or about the body of an animal.

The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, transdermal, subdermal, and/or intraperitoneal administration. In this regard, the compositions of the present invention may be formulated in one or more pharmaceutically acceptable vehicles, including for example sterile aqueous media, buffers, diluents, etc. For example, a given dosage of active ingredient(s) may be dissolved in a particular volume of an isotonic solution (e.g., an isotonic NaCl-based solution), and then injected at the proposed site of administration, or further diluted in a vehicle suitable for intravenous infusion (see, e.g., “REMINGTON'S PHARMACEUTICAL SCIENCES” 15^{th} Ed., pp. 1035-1038 and 1570-1580). While some variation in dosage will necessarily occur depending on the condition of the subject being treated, the extent of the treatment, and the site of administration, the person responsible for administration will nevertheless be able to determine the correct dosing regimens appropriate for the individual subject using ordinary knowledge in the medical and pharmaceutical arts.

Sterile injectable compositions may be prepared by incorporating the disclosed chemotherapeutic delivery system formulations in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The compositions disclosed herein may also be formulated in a neutral or salt form.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, dosage regimen, formulation, and administration of chemotherapeutics disclosed herein will be within the purview of the ordinary-skilled artisan having benefit of the present teaching. It is likely, however, that the administration of a therapeutically-effective (i.e., a pharmaceutically-effective, chemotherapeutically-effective, or an anticancer-effective) amount of the disclosed micro/nano composite drug delivery formulations may be achieved by a single administration, such as, without limitation, a single injection of a sufficient quantity of the delivered agent to provide the desired benefit to the patient undergoing such a procedure. Alternatively, in other circumstances, it may be desirable to provide multiple, or successive administrations of micro/nano composite drug delivery formulations disclosed herein, over relatively short or even relatively prolonged periods, as may be determined by the medical practitioner overseeing the administration of such compositions to the selected individual.

Typically, the micro/nano composite drug delivery formulations described herein will contain at least a chemotherapeutically-effective amount of a first active agent. Preferably, the formulation may contain at least about 0.001% of each active ingredient, preferably at least about 0.01% of the active ingredient, although the percentage of the active ingredient(s) may, of course, be varied, and may conveniently be present in amounts from about 0.01 to about 90 weight % or volume %, or from about 0.1 to about 80 weight % or volume %, or more preferably, from about 0.2 to about 60 weight % or volume %, based upon the total formulation. Naturally, the amount of active compound(s) in each composition may be prepared in such a way that a suitable dosage will be obtained in any given unit dose of the compound. Factors such as solubility, bioavailability, biological t_{1/2}, route of administration, product shelf life, as well as other pharmacological considerations will be contemplated by one of ordinary skill in the art of preparing such pharmaceutical formulations, and as such, a variety of dosages and treatment regimens may be desirable.

Administration of the micro/nano composite drug delivery formulations disclosed herein may be administered by any effective method, including, without limitation, by parenteral, intravenous, intramuscular, or even intraperitoneal administration as described, for example, in U.S. Pat. Nos. 5,543,158; 5,641,515; and 5,399,363 (each of which is specifically incorporated herein in its entirety by express reference thereto). Solutions of the active compounds as free-base or pharmacologically acceptable salts may be prepared in water suitably mixed with a surfactant, such as hydroxypropylcellulose, or other similar fashion. The pharmaceutical forms adapted for injectable administration include sterile aqueous solutions or dispersions, and sterile powders for the extemporaneous preparation of sterile injectable solutions or dispersions including without limitation those described in U.S. Pat. No. 5,466,468 (specifically incorporated herein in its entirety by express reference thereto). In all cases, the form must be sterile and must be fluid to the extent that easy syringability exists. It must be at least sufficiently stable under the conditions of manufacture and storage, and must be preserved against the contaminating action of microorganisms, such as viruses, bacteria, fungi, and such like.

Exemplary carrier(s) may include, for example, a solvent or dispersion medium, including, without limitation, water, ethanol, polyol (e.g., glycerol, propylene glycol, and liquid polyethylene glycol, and the like, or a combination thereof), one or more vegetable oils, or any combination thereof, although additional pharmaceutically-acceptable components may be included.

Proper fluidity of the pharmaceutical formulations disclosed herein may be maintained, for example, by the use of a coating, such as e.g., a lecithin, by the maintenance of the required particle size in the case of dispersion, by the use of a surfactant, or any combination of these techniques. The inhibition or prevention of the action of microorganisms can be brought about by one or more antibacterial or antifungal agents, for example, without limitation, a paraben, chlorobutanol, phenol, sorbic acid, thimerosal, or the like. In many cases, it will be preferable to include an isotonic agent, for example, without limitation, one or more sugars or sodium chloride, or any combination thereof. Prolonged absorption of the injectable compositions can be brought about by the use in the compositions of agents delaying absorption, for example without limitation, aluminum monostearate, gelatin, or a combination thereof.

While systemic administration is contemplated to be effective in many embodiments of the invention, it is also contemplated that formulations disclosed herein be suitable for direct injection into one or more organs, tissues, or cell types in the body. Direct organ administration of the disclosed micro/nano composite drug delivery formulations may be conducted using suitable means, including those known to those of ordinary skill in the oncological arts.

The pharmaceutical micro/nano composite drug delivery formulations disclosed herein are not in any way limited to use only in humans, or even to primates, or mammals. In certain embodiments, the methods and micro/nano composite drug delivery formulations disclosed herein may be employed using avian, amphibian, reptilian, or other animal species. In preferred embodiments, however, the micro/nano composite drug delivery compositions of the present invention are preferably formulated for administration to a mammal, and in particular, to humans, as part of an oncology regimen for treating one or more cancers, such as various lung cancers. The micro/nano composite drug delivery formulations disclosed herein may also be acceptable for veterinary administration, including, without limitation, to selected livestock, exotic or domesticated animals, companion animals (including pets and such like), non-human primates, as well as zoological or otherwise captive specimens, and the like.

### BRIEF DESCRIPTION OF THE DRAWINGS

The following drawings form part of the present specification and are included to demonstrate certain aspects of the present invention. The patent or application file contains at least one drawing executed in color. Copies of this patent or patent application publication with color drawing(s) will be provided by the Office upon request and payment of the necessary fee.

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates. The invention may be better understood by reference to the following description taken in conjunction with the accompanying drawings, in which like reference numerals identify like elements, and in which:

FIG. 1A and FIG. 1B show the synthesis and release schematic of an exemplary enzyme-stimuli multistage vector (ESMSV) in accordance with one aspect of the present. FIG. 1A: MMP2 substrate (SEQ ID NO:1) was conjugated to PLGA-PEG NPs, these PLGA-PEG-Peptide NPs were conjugated to the surface of mesoporous silicon microdisks with DOPC liposomes inside the pores. Coumarin 6 was encapsulated in the PLGA NPs as a model of hydrophobic therapeutics. Negative AF555-tagged siRNA was encapsulated in the DOPC liposomes as a model of siRNA therapeutics. FIG. 1B: Second-stage particles of conjugated PLGA NPs were stimuli-released from the first-stage particles of silicon microdisks once there were MMP2 enzymes;

FIG. 2A, FIG. 2B, FIG. 2C, FIG. 2D, and FIG. 2E show the characterization of an exemplary ESMSV in accordance with one aspect of the present invention. FIG. 2A and FIG. 2B: Scanning electron micrograph (SEM) images of front and back of first-stage silicon microdisks. FIG. 2C and FIG. 2D: SEM images of the front and back of ESMSV. FIG. 2E: Confocal laser scanning microscopy (CLSM) images of exemplary ESMSV. Basal, middle, and top planes of the ESMSV, columns from left to right showing the FITC channel of coumarin 6-loaded PLGA-peptide NPs, the TRITC channel of AF555 siRNA-loaded liposomes, bright field channel of the ESMSV, the merged channel of FITC, TRITC and bright field, respectively;

FIG. 3A, FIG. 3B, and FIG. 3C show the characterization of peptide- (MMP2 substrate) conjugated PLGA-PEG NPs. FIG. 3A: DLS size distribution of PLGA-PEG NPs and PLGA-PEG-peptide NPs. FIG. 3B: XPS analysis of PLGA NPs before and after conjugation of the peptide. FIG. 3C: Zeta-potential of ESMSV and other particles;

FIG. 4 shows the stimuli release of an exemplary ESMSV. The ESMSV released most of the coumarin 6 within 6 hrs of MMP2 enzyme-stimulated release of PLGA NPs;

FIG. 5 shows the cellular uptake efficiency of an exemplary ESMSV and a non-stimuli release MSV control vector in A375 human melanoma cells. Cell culture medium contained 2 μg/mL of MMP2 enzyme;

FIG. 6A and FIG. 6B show the cellular uptake of an exemplary ESMSV and a non-stimuli release MSV control vector in A375 human melanoma cells. The nuclei of A375 cells were stained by DAPI; scale bar=20 μm; Cell culture medium contained 2 μg/mL of MMP2 enzyme;

FIG. 7A, FIG. 7B, FIG. 7C, and FIG. 7D show MMP2 excretion of A375 melanoma cells. FIG. 7A: mRNA expression of MMP2 and MMP9 in HUVEC and A375 cells. FIG. 7B: Protein expression of MMP2 and MMP9 in A375 cell culture medium. FIG. 7C: MMP2 expression in lung tissues with melanoma metastasis. The tumor sections were stained by immunohistochemistry. Scale bar is 50 μm. FIG. 7C and FIG. 7D: Detection of MMP2 expression in melanoma lung metastasis; sections were stained by immunohistochemistry and imaged at either 4× (FIG. 7C) or 40× (FIG. 7D) magnification;

FIG. 8A and FIG. 8B show flow cytometric analysis of percentages of different particles in lung cells and tumor cells 24 hrs after injection of the ESMSV and non-stimuli MSV. In each panel, left is coumarin-negative zone and right is coumarin-positive zone. Lung cell populations and A375 human melanoma cell populations were differentiated by APC-Cy7 anti-human HLA-ABC antibody;

FIG. 9A and FIG. 9B show the design of an exemplary micro/nano composite (MNC) in accordance with one aspect of the present invention. FIG. 9A shows a schematic illustration of an exemplary MNC fabrication process. Porous silicon microdisks were modified with 3-aminopropyltriethoxysilane (APTES), and then loaded with small interfering RNA (siRNA)-containing 1,2-dioleoyl-sn-glycero-3-phosphocholine (DOPC) liposomes through sonication. Docetaxel-encapsulated poly(lactide-co-glycolide) (PLGA)-polyethylene glycol (PEG) nanoparticles were then conjugated to the surfaces of the silicon microdisks. FIG. 9B: Schematic illustration of a therapeutic use of the resulting MNC in accordance with one aspect of the present disclosure. The MNC was designed to metastatic melanoma lesions in the lungs following intravenous injection. Gradual degradation of the silicon material of the first-stage particles triggered the release of liposomes and polymeric second-stage nanoparticles, which then entered cancer cells via endocytosis. EDC=1-ethyl-3-(3-dimethylaminopropyl) carbodiimide; sulfo-NHS=N-hydroxysulfosuccinimide;

FIG. 10A, FIG. 10B, FIG. 10C, FIG. 10D, FIG. 10E, FIG. 10F, FIG. 10G, FIG. 10H, FIG. 10I, FIG. 10J, and FIG. 10K show the characterization of exemplary MNC in accordance with one aspect of the present disclosure. FIG. 10A, FIG. 10B, and FIG. 10C: SEM images of an exemplary MNC. FIG. 10A: Front side (top) and backside (bottom) of a porous silicon microdisk. FIG. 10B: Front-side (top) and backside (bottom) of a porous silicon microdisk coated with PLGA-PEG nanoparticles; FIG. 10C: Front-side (top) and backside (bottom) of a liposome-loaded porous silicon microdisk coated with PLGA-PEG nanoparticles. Scale bar=0.6 FIG. 10D: Chemical structure of the PLGA-PEG polymer. FIG. 10E: SEM images of PLGA-PEG nanoparticles. Scale bar=0.4 FIG. 10F: Size distribution of PLGA-PEG nanoparticles. FIG. 10G and FIG. 10H: Cumulative docetaxel release from the MNC. FIG. 10G: Full-release profile from Day 0 to Day 5; FIG. 10H: 24-hr release profile. FIG. 10I and FIG. 10J: Cumulative siRNA release from the MNC. Full release profile release from Day 0 to Day 12. FIG. 10I: 24-hr release profile. FIG. 10J: Release profile from Day 1 to Day 12. Results are presented as mean±SD of three measurements. FIG. 10K: Western hybridization analysis showing the transfection efficiency of a commercial transfection reagent (INTERFERin®), DOPC liposomes, and MNC. Glyceraldehyde-3-phosphate dehydrogenase (GAPDH) was used as a loading control. Values above the bands represent the relative reduction in protein amount; Lip=DOPC liposomes; MEK=mitogen-activated protein kinase; p-MEK=phosphorylated MEK; Scr=scrambled siRNA;

FIG. 11A and FIG. 11B show the co-localization of liposomes and PLGA-PEG nanoparticles. FIG. 11A and FIG. 11B: Confocal laser scanning microscopy (CLSM) images of MNC. FIG. 11A: Columns from left to right show coumarin 6-loaded PLGA-PEG nanoparticles (green), AF555 siRNA-loaded liposomes (red), brightfield images of the MNC, and co-localization of liposomes and PLGA-PEG nanoparticles. FIG. 11B Uptake of MNC in A375 human melanoma cells; nuclei were stained with 4′,6-diamidino-2-phenylindole (DAPI); scale bar=30 μm;

FIG. 12 shows the biodegradation of an exemplary micro/nano composite (MNC) in phosphate buffered saline (PBS) at 37° C.;

FIG. 13A, FIG. 13B, and FIG. 13C are confocal laser scanning electron micrographs of a MNC positioned on its side. Columns from left to right show: coumarin 6 (green)-loaded PLGA-PEG nanoparticles in the FITC channel; AF555-labeled siRNA (red)-loaded liposomes in the TRITC channel; MNC in the brightfield channel; and MNC in the FITC, TRITC, and brightfield channels;

FIG. 14A, FIG. 14B, FIG. 14C, FIG. 14D, and FIG. 14E show the synergistic therapeutic efficacy of MNC in vitro. FIG. 14A, FIG. 14B, and FIG. 14C: Viability of A375 human melanoma cells exposed to MNCs loaded with the following ratios of BRAF siRNA-to-docetaxel (Doc): 1:4.2 (FIG. 14A), 1:1.1 (FIG. 14B), and 1:0.6 (FIG. 14C), respectively. Data are presented as means±SD of six replicates. FIG. 14D: Combination index for the MNC. FIG. 14E: Western blot analysis showing the effect of the MNC on MEK and extracellular signal-regulated kinase (ERK) protein levels in response to MNCs (siRNA, 100 nM; docetaxel, 100 nM). GAPDH was used as a loading control. Values above the bands represent the relative reduction in protein amount; NP=PLGA-PEG nanoparticles; p-ERK=phosphorylated-ERK;

FIG. 15A, FIG. 15B, FIG. 15C, FIG. 15D, FIG. 15E, FIG. 15F, FIG. 15G, and FIG. 15H show the biodistribution of the MNC in nude mice bearing A375SM melanoma lung metastases. FIG. 15A: Biodistribution of the MNC loaded with DOPC liposomes containing either AF647-labeled siRNA or PLGA-PEG nanoparticles containing lipophilic carbocyanine dye (DiR; 1,1′-dioctadecyl-3,3,3′,3′-tetramethyl indotricarbocyanine iodide). Top lane (left to right): heart, liver, and spleen; bottom lane (left to right): lungs and kidneys. FIG. 15B: Quantitative biodistribution of MNCs loaded with liposomes. FIG. 15C: Quantitative biodistribution of MNCs loaded with PLGA-PEG nanoparticles. FIG. 15D: Ratios of lung-to-liver and liver-to-spleen accumulation of MNCs. Results are presented as mean±SD (n=3). FIG. 15E, FIG. 15F, FIG. 15G, and FIG. 15H: Flow cytometric analysis of various particles in lung tissue (24-hrs' post-injection). A375 human melanoma cells were differentiated from mouse cells using the APC-Cy7-labeled human leukocyte antigen (HLA)-ABC antibody. FIG. 15E: AF555-siRNA-loaded DOPC liposomes. FIG. 15F: MNC with AF555-siRNA-loaded DOPC liposomes. FIG. 15G: Coumarin 6-loaded PLGA-PEG nanoparticles. FIG. 1511: MNC with coumarin 6-loaded PLGA-PEG nanoparticles. SSC=side scatter;

FIG. 16 shows the anticancer activity of MNC in nude mice bearing A375SM-Luc melanoma lung metastases. Treatment was administered intravenously weekly for four weeks (docetaxel: 4 mg/kg; BRAF siRNA: 1 mg/kg). Therapeutic efficacy was assessed via bioluminescent imaging. Results are presented as mean±SD (n=5). *, p<0.5; **,p<0.01 (t-test); Lip=liposome;

FIG. 17 shows the DNA sequence analysis of A375 cells (A375), highly metastatic A375 cells (A375SM) and A375SM cells with luciferase expression (A375SM-Luc) (SEQ ID NO:8). The data reveals the presence of the BRAF V600E mutation in the cell lines; WT=wild type (SEQ ID NO:9); CONSENSUS sequence (SEQ ID NO:10);

FIG. 18 shows the cell viability of A375 melanoma cells treated with various BRAF V600E siRNAs (100 nM) for 72 hr. The commercial transfection reagent INTERFERin® was used for transfection. Results are presented as the mean±SD of six replicates;

FIG. 19A, FIG. 19B, FIG. 19C, FIG. 19D, FIG. 19E, and FIG. 19F show the anticancer efficacy of the MNC in vivo. FIG. 19A: Anticancer efficacy of the MNC in a lung metastasis mouse model of A375SM human melanoma cells. Animals received weekly intravenous injections of the MNC for four weeks (docetaxel, 4 mg/kg; BRAF siRNA, 1 mg/kg). Melanoma cells were transfected with a luciferase gene and therapeutic efficacy was assessed through real-time monitoring of bioluminescence. FIG. 19B: Survival curves of mice bearing melanoma lung metastasis (n=8; Log-rank test, P<0.0001). Day 0 indicates treatment initiation. FIG. 19C: Number of pulmonary surface metastases 35 days after treatment initiation. Data is presented as mean±SD (n=4). FIG. 19D: Images of lungs 35 days after initiation of treatment. FIG. 19E: Histological sections of the lungs 35 days after initiation of treatment. Tissues were stained with hematoxylin and eosin (H&E). Scale bar=200 μm. FIG. 19F: Immunohistochemistry of BRAF in melanoma lung metastases 35 days after treatment initiation. Scale bar=50 μm;

FIG. 20A, FIG. 20B, and FIG. 20C show histological images of the lungs 35 days after treatment initiation. Treatment was administered intravenously once a week for four weeks (docetaxel: 4 mg/kg, BRAF siRNA: 1 mg/kg). Tissues were stained with H&E. Scale bar=200 μm;

FIG. 21 shows the cell viability of A375 cells and A375SM-Luc cells after treatment with MNCs containing various concentrations of BRAF siRNA (Braf) and docetaxel (Doc) for 72 hr. Results are presented as mean±SD of six replicates;

FIG. 22A and FIG. 22B show the biodistribution of particles in nude mice bearing A375SM-Luc melanoma lung metastasis. FIG. 22A: (left to right): heart, liver, and spleen. FIG. 22B: (left to right): lungs and kidneys;

FIG. 23 shows mouse body weights. Day 0 represents treatment initiation. Treatment was administered intravenously once a week for four weeks (docetaxel: 4 mg/kg, BRAF siRNA: 1 mg/kg). Results are presented as mean±SD (n=8);

FIG. 24A, FIG. 24B, and FIG. 24C show the number of pulmonary surface metastases 22 days after treatment initiation (FIG. 24A). Cancer cells were injected into six-week-old mice (10⁶ cells/mouse). Data is presented as the mean±SD (n=4). FIG. 24B and FIG. 24C: Normalized number of lung metastases. Cancer cells were injected into ten-week-old (FIG. 24B) and six-week-old (FIG. 24C) mice, respectively (10⁶ cells/mouse). In all cases, treatment was initiated two weeks after cancer cell injection; and

FIG. 25 shows a proposed mechanism for therapeutic synergy of the disclosed compositions. In the in vitro diagram, numbers represent: inhibition of BRAF by siRNA (1), possible mechanisms of resistance to BRAF inhibition (2), inhibition of mitogen-activated protein kinase kinase 1/2 (MEK1/2) by docetaxel (3), mechanism of resistance to docetaxel, and (4) EPR, enhanced permeability and retention; ERK, extracellular-signal-regulated kinase; MAPK, mitogen activated kinase.

### BRIEF DESCRIPTION OF THE SEQUENCES

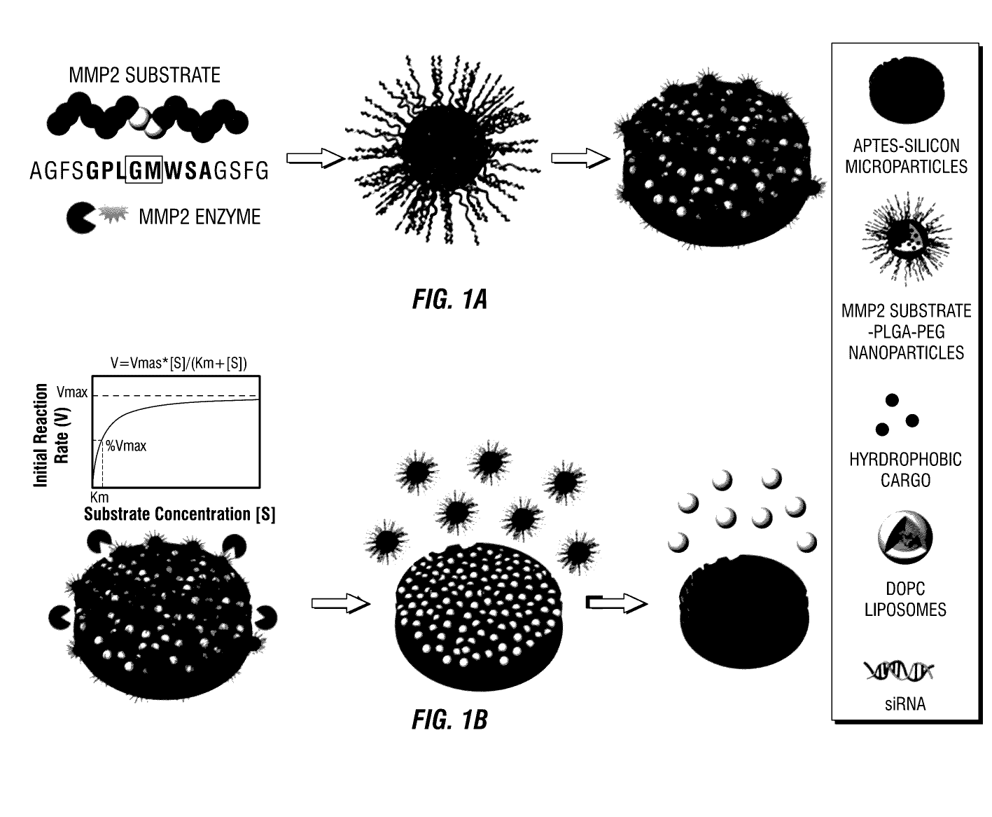

SEQ ID NO:1 is an exemplary MMP2 substrate peptide depicted in FIG. 1A, having the amino acid sequence AGFSGPLGMWSAGSFG, as used and described in accordance with various aspects of the present disclosure;

SEQ ID NO:2 is an exemplary BRAF-specific DNA oligonucleotide forward primer (5′-GCATCTCACCTCATCCTAACAC-3′), as used and described in accordance with various aspects of the present disclosure;

SEQ ID NO:3 is an exemplary BRAF-specific DNA oligonucleotide reverse primer (5′-CTAGTAACTCAGCAGCATCTCA-3′), as used and described in accordance with various aspects of the present disclosure;

SEQ ID NO:4 is an exemplary BRAF sequencing primer (5′-GCATCTCACCTCATCCTAACAC-3′), as used and described in accordance with various aspects of the present disclosure;

SEQ ID NO:5 is an exemplary BRAF V600E siRNA sequence (5′-GCUACAGAGAAAUCUCGAU-3′), as used and described in accordance with various aspects of the present disclosure;

SEQ ID NO:6 is an exemplary BRAF V600E siRNA sequence (5′-AACAGUCUACAAGGGAAAGUG-3′), as used and described in accordance with various aspects of the present disclosure;

SEQ ID NO:7 is an exemplary BRAF V600E siRNA sequence (5′-GCUACAGAGAAAUCUCGAU-3′), as used and described in accordance with various aspects of the present disclosure;

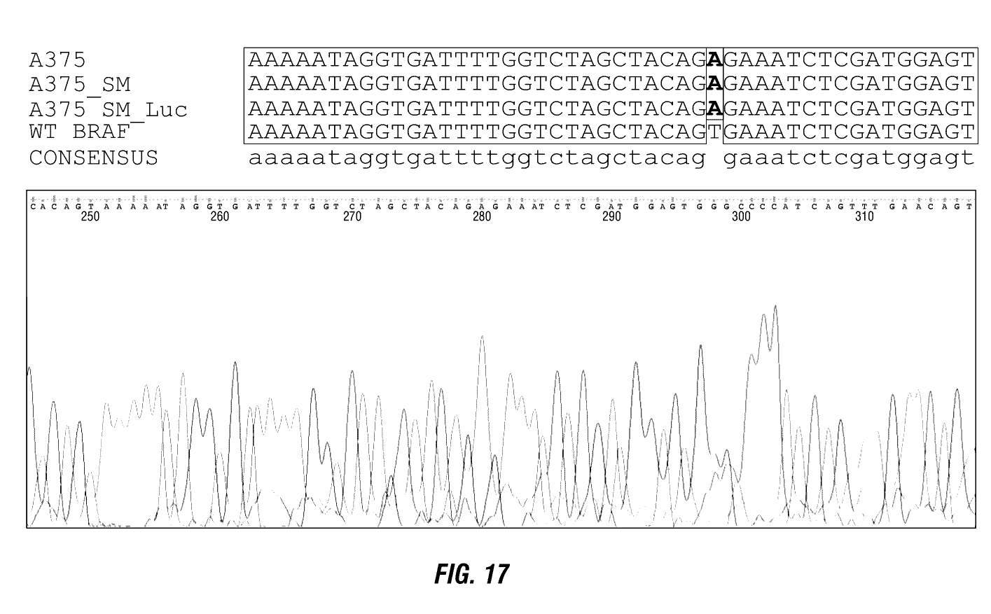

SEQ ID NO:8 is an exemplary nucleic acid sequence depicted as “A375,” “A375_SM,” and “A375 SM_Luc,” in FIG. 17, having the nucleotide sequence: 5 ′-AAAAATAGGTGATTTTGGTCTAGCTACAGAGAAATCTCGATGGAGT-3 ; as used and described in accordance with various aspects of the present disclosure;

SEQ ID NO:9 is an exemplary nucleic acid sequence depicted as “WT BRAF” in FIG. 17, having the sequence: 5′-AAAAATAGGTGATTTTGGTCTAGCTACAGAG AAATCTCGATGGAGT-3′, as used and described in accordance with various aspects of the present disclosure; and

SEQ ID NO:10 is an exemplary nucleic acid consensus sequence depicted as “CONSENSUS” in FIG. 17, having the sequence: 5′-AAAAATAGGTGATTTT GGTCTAGCTACAGGAAATCTCGATGGAGT-3′, as used and described in accordance with various aspects of the present disclosure.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

### Pharmaceutical Formulations

The pharmaceutical formulations of the present invention may further comprise one or more excipients, buffers, or diluents that are particularly formulated for administration to a human patient. Compositions may further optionally comprise one or more additional microspheres, microparticles, nanospheres, or nanoparticles, and may be formulated for administration to one or more cells, tissues, organs, or body of a human undergoing treatment for one or more types of cancer, including lung cancers, such as melanoma metastasis to the lung, in particular.

Formulation of pharmaceutically-acceptable excipients and carrier solutions is well-known to those of ordinary skill in the art, as is the development of suitable dosing and treatment regimens for using the particular compositions described herein in a variety of treatment regimens, including e.g., without limitation, oral, parenteral, intravenous, intranasal, intratumoral, and intramuscular routes of administration.

Typically, the chemotherapeutic micro/nano particle delivery systems and compositions comprising them may be formulated to contain at least about 0.1% of at least a first active compound or more, although the percentage of the active ingredient(s) may, of course, be varied and may conveniently be between about 1 or 2% and about 70% or 80% or more of the weight or volume of the total formulation. Naturally, the amount of active compound(s) in each composition may be prepared is such a way that a suitable dosage of the therapeutic agent will be obtained in any given unit dose of the chemotherapeutic formulations disclosed herein. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The particular amount of compositions employed, and the particular time of administration, or dosage regimen for compositions employing the disclosed micro/nano particle delivered chemotherapeutic formulations will be within the purview of a person of ordinary skill in the art having benefit of the present teaching. It is likely, however, that the administration of diagnostically- and/or therapeutically-effective amounts of the disclosed formulations may be achieved by administration of one or more doses of the formulation, during a time effective to provide the desired chemotherapeutic benefit to the patient undergoing such treatment. Such dosing regimens may be determined by the medical practitioner overseeing the administration of the chemotherapeutics, depending upon the particular condition or the patient, the extent of the cancer, etc.

Typically, formulations of the active ingredients in the disclosed compositions will contain an effective amount for the particular therapy regimen of a given patient. Preferably, the formulation may contain at least about 0.1% of each active ingredient, although the percentage of the active ingredient(s) may, of course, be varied, and may conveniently be present in amounts from about 0.5 to about 80 weight % or volume %, or from about 1 to about 70 weight % or volume %, or more preferably, from about 2 to about 50 weight % or volume %, based upon the total formulation. Naturally, the amount of active compound(s) may be prepared in such a way that a suitable dosage will be obtained in any given unit dose of the compound. Factors such as solubility, bioavailability, biological t_{1/2}, route of administration, product shelf life, as well as other pharmacological considerations will be contemplated by one of ordinary skill in the art of preparing such pharmaceutical formulations, and as such, a variety of dosages and treatment regimens may be desirable.

Compositions for the Preparation of Medicaments

Another important aspect of the present invention concerns methods for using the disclosed compositions (as well as formulations including them) in the preparation of medicaments for treating or ameliorating the symptoms of various diseases, dysfunctions, or deficiencies in an animal, such as a vertebrate mammal. Use of the disclosed compositions is particular contemplated in the chemotherapeutic treatment of one or more types of cancer in a human, and particularly in the treatment of lung cancers, such as melanoma metastasis lung cancer, in a human.

Such use generally involves administration to the mammal in need thereof one or more of the disclosed micro/nano particle two-stage chemotherapeutic delivery system compositions, in an amount and for a time sufficient to treat, lessen, or ameliorate one or more symptoms of the cancer in the affected mammal.

Pharmaceutical formulations including one or more of the disclosed micro/nano particle two-stage chemotherapeutic delivery systems also form part of the present invention, and particularly those compositions that further include at least a first pharmaceutically-acceptable excipient for use in the therapy or amelioration of one or more symptoms of mammalian lung cancer, and particularly, for use in the therapy or amelioration of one or more symptoms of melanoma metastasis lung cancer in a human.

Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs.

The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long-standing patent law convention, the words “a” and “an,” when used in this application (including in the appended claims), denotes “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example, from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s), or such like, or a combination thereof that is pharmaceutically acceptable for administration to the relevant animal or acceptable for a therapeutic or diagnostic purpose, as applicable.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, e.g., plasmids, cosmids, phage, viruses, and the like.

The term “effective amount,” as used herein, refers to an amount that is capable of treating or ameliorating a disease or condition or otherwise capable of producing an intended therapeutic effect.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous polynucleotide segments introduced through the hand of man.

As used herein, the term “epitope” refers to that portion of a given immunogenic substance that is the target of (i.e., is bound by), an antibody or cell-surface receptor of a host immune system that has mounted an immune response to the given immunogenic substance as determined by any method known in the art. Further, an epitope may be defined as a portion of an immunogenic substance that elicits an antibody response or induces a T-cell response in an animal, as determined by any method available in the art (see, e.g., Geysen et al., 1984). An epitope can be a portion of any immunogenic substance, such as a protein, polynucleotide, polysaccharide, an organic or inorganic chemical, or any combination thereof. The term “epitope” may also be used interchangeably with “antigenic determinant” or “antigenic determinant site.”

The term “for example” or “e.g.,” as used herein, is used merely by way of example, without limitation intended, and should not be construed as referring only those items explicitly enumerated in the specification.

As used herein, “heterologous” is defined in relation to a predetermined referenced DNA or amino acid sequence. For example, with respect to a structural gene sequence, a heterologous promoter is defined as a promoter that does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polypeptides or polynucleotides, sequences that have the same essential structure, despite arising from different origins. Typically, homologous proteins are derived from closely related genetic sequences, or genes. By contrast, an “analogous” polypeptide is one that shares the same function with a polypeptide from a different species or organism, but has a significantly different form to accomplish that function. Analogous proteins typically derive from genes that are not closely related.

As used herein, the term “homology” refers to a degree of complementarity between two polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state. Thus, an isolated peptide in accordance with the invention preferably does not contain materials normally associated with that peptide in its in situ environment.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed compositions, such as, for example, in a laboratory or clinical application.

“Link” or “join” refers to any method known in the art for functionally connecting two or more entities, including, for example, two or more peptides (including, without limitation, recombinant fusion proteins), or two or more components of a hybrid composite drug delivery vehicle, using a method known in the art, including, without limitation, by covalent bonding, disulfide bonding, ionic bonding, hydrogen bonding, electrostatic bonding, or a combination thereof.

As used herein, “mammal” refers to the class of warm-blooded vertebrate animals that have, in the female, milk-secreting organs for feeding the young. Mammals include without limitation humans, apes, many four-legged animals, whales, dolphins, and bats. A human is a preferred mammal for purposes of the invention.

“Microparticle” means a particle having a maximum characteristic size from 1 micron to 1000 microns or from 1 micron to 100 microns. Preferably, the porous particle of this disclosure should have a relatively high porosity to enable loading of the polymeric-active agent conjugate in the pores of the porous particles. Optionally, the porous particles of the present disclosure may be coated with a targeting moiety. Such embodiments may be useful for targeted delivery of the active compound to the desired disease site.

“Nanoparticle” means a particle having a maximum characteristic size of less than 1 micron. Preferably, the polymeric-active agent conjugate of this disclosure forms nanoparticles upon release from the porous silicon particle upon physiological degradation of the porous particle, and upon coming in contact with an aqueous environment.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites).

The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snoRNA), small nuclear RNA (snRNA), small temporal RNA (stRNA), and the like, as well as any combinations thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases, and intronic sequences may be of variable lengths; some polynucleotide elements may be operably linked, but not contiguous.

As used herein, the term “patient” (also interchangeably referred to as “host” or “subject”) refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a “patient” refers to any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, and any animal under the care of a veterinary practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′ dibenzylethylenediamine or ethylenediamine; and combinations thereof.

The term “pharmaceutically acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to all amino acid chain lengths, including those of short peptides of from about 2 to about 20 amino acid residues in length, oligopeptides of from about 10 to about 100 amino acid residues in length, and polypeptides including about 100 amino acid residues or more in length.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found(e.g., cellular material such as cellular proteins, peptides, nucleic acids, etc.).

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment,” as used herein, refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “sequence,” when referring to amino acids, relates to all or a portion of the linear N-terminal-to-C-terminal order of amino acids within a given amino acid chain, e.g., polypeptide or protein; “subsequence” means any consecutive stretch of amino acids within a sequence, e.g., at least 3 consecutive amino acids within a given protein or polypeptide sequence. With reference to nucleotide and polynucleotide chains, “sequence” and “subsequence” have similar meanings relating to the 5′-to-3′ order of nucleotides.

The phrase a “sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X.

The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

Suitable standard hybridization conditions for the present invention include, for example, hybridization in 50% formamide, 5x Denhardt's solution, 5x SSC, 25 mM sodium phosphate, 0.1% SDS and 100 μg/mL of denatured salmon sperm DNA at 42° C. for 16 hr followed by 1-hr sequential washes with 0.1x SSC, 0.1% SDS solution at 60° C. to remove the desired amount of background signal. Lower stringency hybridization conditions for the present invention include, for example, hybridization in 35% formamide, 5x Denhardt's solution, 5x SSC, 25 mM sodium phosphate, 0.1% SDS and 100 μg/mL denatured salmon sperm DNA or E. coli DNA at 42° C. for 16 hr followed by sequential washes with 0.8x SSC, 0.1% SDS at 55° C. Those of ordinary skill in the art will recognize that conditions can be readily adjusted to obtain the desired level of stringency.

Naturally, the present invention also encompasses nucleic acid segments that are complementary, essentially complementary, and/or substantially complementary to at least one or more of the specific nucleotide sequences specifically set forth herein. Nucleic acid sequences that are “complementary” are those that are capable of base pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to one or more of the specific nucleic acid segments disclosed herein under relatively stringent conditions such as those described immediately above.

As described above, the probes and primers of the present invention may be of any length. By assigning numeric values to a sequence, for example, the first residue is 1, the second residue is 2, etc., an algorithm defining all probes or primers contained within a given sequence can be proposed:

n to n+y,

where n is an integer from 1 to the last number of the sequence and y is the length of the probe or primer minus one, where n+y does not exceed the last number of the sequence.

Thus, for a 25-basepair probe or primer (i.e., a “25-mer”), the collection of probes or primers correspond to bases 1 to 25, bases 2 to 26, bases 3 to 27, bases 4 to 28, and so on over the entire length of the sequence. Similarly, for a 35-basepair probe or primer (i.e., a “35-mer), exemplary primer or probe sequence include, without limitation, sequences corresponding to bases 1 to 35, bases 2 to 36, bases 3 to 37, bases 4 to 38, and so on over the entire length of the sequence. Likewise, for 40-mers, such probes or primers may correspond to the nucleotides from the first basepair to bp 40, from the second by of the sequence to bp 41, from the third bp to bp 42, and so forth, while for 50-mers, such probes or primers may correspond to a nucleotide sequence extending from bp 1 to bp 50, from bp 2 to bp 51, from bp 3 to bp 52, from bp 4 to bp 53, and so forth.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will preferably be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid or polypeptide sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

The term “therapeutically practical time period” means a time necessary for an active agent to be therapeutically effective. The term “therapeutically-effective” refers to a reduction in the severity and/or frequency of one or more symptoms, an elimination of symptoms, and/or one or more underlying causes, the prevention of an occurrence of one or more symptoms and/or their underlying cause, and/or an improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally-occurring, or produced by synthetic or recombinant methods, or any combination thereof. Drugs that are affected by classical multidrug resistance, such as the vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and to Goodman and Gilman's “Pharmacological Basis of Therapeutics” tenth edition, Hardman et al. (Eds.) (2001).

“Transcriptional regulatory element” refers to a polynucleotide sequence that activates transcription alone or in combination with one or more other nucleic acid sequences. A transcriptional regulatory element can include, for example, one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA foot-printing, gel mobility shift assays, and the like, and/or can be predicted based on known consensus sequence motifs, or by other methods known to those of ordinary skill in the art.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

As used herein, the term “transformed cell” is intended to mean a host cell whose nucleic acid complement has been altered by the introduction of one or more exogenous polynucleotides into that cell.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The expression “zero-order or near-zero-order” as applied to the release kinetics of the active agent delivery composition disclosed herein is intended to include a rate of release of the active agent in a controlled manner over a therapeutically practical time period following administration of the composition, such that a therapeutically effective plasma concentration of the active agent is achieved.

The section headings used throughout are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure, appreciate that many changes can be made in the specific embodiments that are disclosed, and still obtain a like or similar result without departing from the spirit and scope of the invention.

### Example 1

### Enzyme-Stimuli Multistage Vector for Transportation of Therapeutics to Tumor Lung Metastasis

Various nano platforms have been reported to release drug by responding to the physicochemical change in organism. The design of these platforms is to solve the release problem of the therapeutics at target cells. In this example, an enzyme-stimuli multistage vector (ESMSV) has been developed to realize the stimuli-release of a second-stage vector from a first-stage vector in lungs with tumor metastasis. Specifically, PLGA nanoparticles were conjugated with matrix metalloproteinase-2 (MMP-2) substrate, and further conjugated to the surface of silicon microparticles. Instead of stimuli-release of drugs, PLGA nanoparticles are released from silicon microparticles triggered by MMP-2. The release of PLGA nanoparticles improved the cellular uptake efficiency. By developing a stimuli-release multistage vector, transportation of therapeutics into cancer cells of lung metastasis can now be enhanced in vivo.

Therapeutics for cancer such as chemotherapeutic drugs, siRNA, and proteins, often suffer from low efficacies in clinical application, partially due to their insufficient accumulation at the targeted cancer lesions, and/or unspecific distribution in various cells and/or tissues. Nanotechnology has developed and been applied to the delivery of chemotherapeutics in an effort to delay clearance from the bloodstream, overcome biological barriers, increase transportation to target lesions, and to decrease the side effects of the therapy. Nevertheless, most nanotechnology-based drug delivery systems still suffer from an indistinctive release in both the bloodstream and in tissues. The unwanted release in the circulatory system decreases the transport of therapeutics to target lesions, and increases unwanted side effects. Meanwhile, release of the active agent from the nanoparticles at the lesions/target site is often too slow to meet the requirements of efficacious therapy.

To solve these problems, the stimuli-response delivery systems described herein were developed and studied, to provide controlled release of the delivered chemotherapeutic agents (both spatially and temporally) according to certain stimuli (either internal or external). These stimuli can include physiological and/or pathological changes at the lesions or target site, such as changes in pH, redox potential, oxidative stress, and the like.

The use of enzymes as a stimulus represents an attractive option for the delivery of chemotherapeutic agents to cancer lesions because enzyme activity is often related to the progression, metastasis, and/or angiogenesis of tumor tissues, and one or more enzymes are often overexpressed in tumor site compared to expression in normal tissues. For example, most mammalian tumors overexpress matrix metalloproteinase (MMP) enzymes, and excrete them. Exploiting this fact, the inventors conjugated an MMP2 substrate peptide between PLGA NPs and MSV forming an enzymatic cleavage site. The MMP2 enzyme can recognize this site, and cleave the peptide so that the PLGA NPs are released from the MSV. This release occurs preferentially in tumor tissues, because of the localized excretion of MMP2 enzyme by the cancer cells.

Matrix metalloproteinases (MMPs) are a family of zinc- and calcium-dependent proteolytic enzymes. They digest various components of the extracellular matrix (ECM), including collagen, laminin, fibronectin, vitronectin, elastin, and proteoglycans. MMPs, such as MMP2 and MMP9, are important to invasion, migration, metastasis, and tumorigenesis, and are thus often highly expressed in tumor tissues. Therefore, nanocarriers responding to MMPs are designed and used for delivery of cancer therapeutics. This kind of nanocarriers is often composed with a peptide sequence that can be recognized and cleaved by the enzyme. For example, Dorresteijn et al. (2014) synthesized PLLA-peptide-PLLA tri-block copolymer nanoparticles in which the peptide was a MMP2 cleavage site. It realized enzyme cleavage-dependent release in vitro without changing the particle morphology. The study has demonstrated the enzyme-response of nanocarriers in vitro.

However, once the situation is considered in vivo, though with the help of EPR effect these enzyme-stimuli nanoparticles can accumulate at tumor tissues, the enzyme-stimuli dissolution of the nanocarriers usually takes place at the ECM outside the cancer cells. The released therapeutics will suffer aggregation, low efficiency of cell penetration, and chemical denaturation without the protection of nanocarriers, which will largely decrease the efficacy of the therapeutics.

To overcome these types of problems, a new and improved delivery system is necessary, in which first-stage particles transport a second-stage particles to selected tumor tissues, and then the second-stage particles are released responsively to the enzymes, and the therapeutics contained therein are then transferred to the selected target cells of interest.

This example describes the development of an ESMSV for transportation of therapeutics to tumor lung metastasis.

Materials and Methods

Materials. PLGA-PEG-COOH was prepared according to a previous report (Cheng et al., 2007). MMP2 substrate peptide having the amino acid sequence AGFSGPLGMWSAGSFG (SEQ ID NO:1) was purchased from Peptide 2.0 (Chantilly, Va., USA). Sulfo-NHS was bought from Thermo-Fisher Scientific, Inc. (Waltham, Mass., USA). DOPC was purchased from Avanti Polar Lipids, Inc. (Alabaster, Ala., USA). Negative AF555-tagged siRNA was purchased from Qiagen (Germantown, Md., USA). PBS, Fetal bovine serum (FBS), DMEM, trypsin, penicillin, and streptomycin were purchased from GE Healthcare Life Sciences (Pittsburgh, Pa., USA). All other chemicals were purchased from Sigma-Aldrich (St. Louis, Mo., USA).

Preparation and Characterization of ESMSV. The enzyme-stimuli multistage vector (ESMSV) was fabricated by conjugating the MMP2 substrate-modified PLGA-PEG nanoparticles to the APTES-modified silicon microdisks. Photolithography and electrochemical etch were used to produce the mesoporous silicon microdisks with the size of 2.6 μm in diameter, 0.7 μm in height and 5060 nm in pore diameter. These silicon microdisks were then modified with 3-aminopropyltriethoxysilane (APTES). The coumarin 6-loaded PLGA-PEG nanoparticles were prepared by the nanoprecipitation method (Fonseca et al., 2002). The PLGA-PEG-COOH NPs were then modified with MMP2 substrate. In brief, 1 mg/mL PLGA-PEG-COOH NPs was dissolved in 10 mL PBS buffer. Then 3 mg EDC and 2.4 mg sulfo-NHS were added to activate the surface functional group of the NPs. MMP2 substrate was added to the solution at a concentration of 0.2 mg/mL. The reaction lasted for 3 hr. After the reaction, MMP2-conjugated PLGA-PEG NPs were washed, and then centrifuged 3 times with water at 10,000 rpm.

To prepare the ESMSV, 20 mg of MMP2-conjugated PLGA-PEG NPs were dissolved in PBS buffer, and then activated by EDC (10 mg) and sulfo-NHS (8 mg) for 30 min. 0.2 billion APTES-modified silicon microdisks were added to the solution with the final volume of 10 mL, and stirred for 3 hr. After the reaction, ESMSVs were washed and centrifuged 3 times with water at 4500 rpm. For the liposome-loaded ESMSV, the liposomes were loaded before the conjugation of MMP2 substrate modified PLGA-PEG NPs. The loading process of liposomes into the pores of MSV was performed according to a previous report (Fine et al., 2013).

The size and zeta potential of the particles were measured (Zetasizer® Nano Range, Malvern Instruments; Worcestershire, UK). The SEM characterization was performed on an ultra-high resolution scanning electron microscope (SEM 230, NovaNano, Hillsboro, Oreg., USA).

Enzyme-Stimuli Release. The coumarin 6-loaded PLGA-NPs (with or without MMP2 substrate) were centrifuged at 1500 rpm for 5 min, and the supernatant containing the nanoparticles was collected. The nanoparticles were then conjugated to silicon microparticles. The micro/nano particles were centrifuged at 1500 rpm for 5 min and the pellet was retained. The collected micro/nano particles in the pellet were then dispersed in 200 μL MMP2 reaction buffer (50 mM HEPES with 10 mM CaCl₂) with or without MMP2 enzyme at a concentration of 2 μg/mL. The dispersion in tubes was put in an orbital shaker (120 rpm) in a water bath at 37° C. At designated time intervals, the tube of the suspension was centrifuged at 1500 rpm for 5 min. The pellets were drained and resuspended in fresh buffer to continue the drug release process. Supernatants were added into black 96-well microplates, and the fluorescent intensity was recorded for both free coumarin 6 and coumarin 6-loaded PLGA NPs. The error bars were obtained from triplicate samples.

Cellular Uptake. The cellular uptake of PLGA-PEG NPs-conjugated silicon microparticles with (MSV/Peptide-NPs) or without (MSV/NPs) MMP2 substrate were compared quantitatively. The A375 cells were seeded into 96-well black plates (Costar, Corning, Keller, Tex., USA) at 5×10³ cells/well (0.1 mL) and after the cells reached 80% confluence, the medium was changed to the medium with coumarin 6-loaded MSV/NPs and MSV/Peptide NPs at concentration of 0.1 billion/mL. The medium contained 2 μg/mL MMP2 enzyme. After incubation of different times, the particles suspension in the testing wells was removed and the wells were washed with 0.1 mL PBS three times to remove the particles outside the cells. After that, 50 μL of 0.5% Triton X-100 in 0.2 N NaOH was added to lyse the cells. A microplate reader (Genios, Tecan; Mannedorf, SWITZERLAND) was used to measure the fluorescence intensity from coumarin-6 loaded particles in the desired wells at an excitation wavelength of 430 nm and an emission wavelength of 485 nm. The cellular uptake efficiency was expressed as the percentage of fluorescence of the test sample over that of the positive control.

Confocal Microscopy. After reaching confluence, A375 cells were detached, counted, and seeded in a 4-well cover glass chamber (LAB-TEK, Nagle Nunc, Ill., USA) at 2×10⁴ cells/well overnight. Then, the medium was replaced by the liposomes (with AF555 siRNA)-loaded, PLGA-PEG NPs (with coumarin 6)-conjugated silicon microparticles with (MSV/Peptide-NPs) or without (MSV/NPs) MMP2 substrate at concentration of 0.1 billion/mL and incubated for 24 hr. The cells were washed twice with pre-warmed 1× PBS, and fixed with 70% ethanol for 20 min. After that, the cells were washed twice with PBS, and then the nuclei were counterstained by DAPI for 30 min. The cells were washed again twice by 1× PBS, and immersed in 1× PBS for confocal microscopic imaging.

Cell Culture. A375 human melanoma cells were obtained from the American Type Culture Collection (ATCC; Manassas, Va., USA).

The cells were cultured in DMEM with 10% (vol.//vol.) fetal bovine serum (FBS) and 1% (vol./vol.) penicillin and streptomycin in an incubator at 37° C. and 5% CO₂. The cells were sub-cultivated at 70% confluence with 0.25% trypsin.

Animal Studies. Animal studies were performed in accordance with the guidelines of the Animal Welfare Act and the Guide for the Care and Use of Laboratory Animals following protocols approved by the Institutional Animal Care and Use Committee (IACUC). Six-week-old female, athymic, nude mice were purchased from Charles River Laboratories (Boston, Mass., USA). A375SM-Luc human melanoma cancer cells with stable expression of luciferase were harvested from exponential cultures and intravenously inoculated into the tail veins of mice at a concentration of 10⁶ cells/mouse. Lung metastases were monitored by a bioluminescence imaging system (IVIS 200; Waltham, Mass., USA). Animals are injected with luciferin potassium salt in PBS buffer by intraperitoneal injection. Then, animals were anesthetized with isoflurane (2-chloro-2-(difluoromethoxy)-1,1,1-trifluoro-ethane) in an anesthetic chamber or via nose cone, and then placed inside the camera (INVENTORS: IS THIS CORRECT?) where anesthesia was maintained via nose cone. Following the imaging session (generally less than 10 min), animals were returned to their cages and observed until normal behavior was restored.

Expression of MMP2 Enzyme In Vitro and In Vivo. The expression of MMP2 mRNA and excreted MMP2 enzyme for A375 cells was analyzed and compared with the expression of MMP9 in vitro. The expression of mRNA was analyzed quantitatively by real time RT-PCR. For testing the expression of excreted MMP2 and MMP9 enzyme, A375 cells were cultured in medium without FBS. After 72-hrs' incubation, the medium was collected and analyzed by silver staining (using the standard protocol from the Pierce™ Silver Stain Kit from Thermo-Fisher Scientific Inc. (Life Technologies, Waltham, Mass., USA).

In brief, the protocol takes the following steps:

Step 1. Wash the polyacrylamide gel in ultrapure water for 5 min. Replace the water and wash for another 5 min.

Step 2. Fix gel in 30% ethanol:10% acetic acid solution (i.e., 6:3:1 water:ethanol:acetic acid) for 15 min. Replace the solution and fix for another 15 min.

Step 3. Wash gel in 10% ethanol for 5 min. Replace solution and wash for another 5 min.

Step 4. Wash gel in ultrapure water for 5 min. Replace water and wash for another 5 min.

Step 5. Prepare Sensitizer Working Solution by mixing 1 part Silver Stain Sensitizer with 500 parts ultrapure water (e.g., mix 50 μL Sensitizer with 25 mL water).

Step 6. Incubate gel in Sensitizer Working Solution for exactly 1 min, and then wash with two changes of ultrapure water for 1 min each.

Step 7. Prepare Stain Working Solution by mixing 1 part Silver Stain Enhancer with 50 parts Silver Stain (e.g., 0.5 mL of enhancer with 25 mL stain).

Step 8. Incubate gel in Stain Working Solution for 30 min. (Gel may be incubated in Stain Working Solution for as short as 5 min or as long as overnight without affecting stain performance).

Step 9. Prepare Developer Working Solution by mixing 1 part Silver Stain Enhancer with 50 parts Silver Stain Developer (e.g., mix 0.5 mL of enhancer with 25 mL developer).

Step 10. Prepare 5% acetic acid solution as a stop solution.

Step 11. Quickly wash gel with two changes of ultrapure water for 20 sec each.

Step 12. Immediately add Developer Working Solution and incubate until protein bands appear (2-3 min). (Protein bands will typically begin to appear within 30 sec and then continue to develop. Between 2 and 3 min, protein detection vs. background is optimal. After 3 min, lane background signal may increase to undesirable levels).

Step 13. When the desired band intensity is reached, replace Developer Working Solution with prepared Stop Solution (5% acetic acid). Wash gel briefly, then replace Stop Solution and incubate for 10 min).

Step 14. Perform standard western blot analysis.

Buffer Preparation:

Step 1. To prepare 1 L of 1× PBS: add 50 mL 20× PBS to 950 mL dH₂O; mix.

Step 2. To prepare 1 L of 1× TBS: add 100 mL 10× to 900 mL dH₂O; mix.

Step 3. To prepare fresh 3× reducing loading buffer by adding 1/10 volume 30× DTT to 1 volume of 3× SDS loading buffer. Dilute to 1× with dH₂O.

Step 4. To prepare 1 L of 1× running buffer: add 100 mL 10× running buffer to 900 mL dH₂O; mix.

Step 5. To prepare 1 L of 1× Transfer Buffer: add 100 mL 10× Transfer Buffer to 200 mL methanol+700 mL dH₂O; mix.

Step 6. To prepare 1 L 1× TBST: add 100 mL 10× TBST to 900 mL dH₂O; mix.

Step 7. To prepare Blocking Buffer: 1× TBST with 5% (wt./vol.) nonfat dry milk; for 150 mL, add 7.5 g nonfat dry milk to 150 mL 1× TBST and mix well.

Step 8. Wash Buffer: 1× TBST.

Step 9. To prepare primary Antibody Dilution Buffer: 1× TBST with 5% BSA or 5% nonfat dry milk as indicated on primary antibody datasheet; for 20 mL, add 1.0 g BSA or nonfat dry milk to 20 mL 1× TBST, and mix well.

### Protein Blotting:

Step 1. Treat cells by adding fresh media containing regulator for desired time.

Step 2. Aspirate media from cultures; wash cells with 1× PBS; aspirate.

Step 3. Lyse cells by adding 1× SDS sample buffer (100 μL per well of 6-well plate or 500 μL for a 10-cm diameter plate). Immediately scrape the cells off the plate and transfer the extract to a microcentrifuge tube. Keep on ice.

Step 4. Sonicate for 10-15 sec to complete cell lysis, and shear DNA to reduce sample viscosity.

Step 5. Heat a 20-μL sample to 95-100° C. for 5 min; cool on ice.

Step 6. Microcentrifuge for 5 min.

Step 7. Load 20 μL onto SDS-PAGE gel (10 cm×10 cm).

Step 8. Electrotransfer to nitrocellulose membrane.

### Membrane Blocking and Antibody Incubation:

Step 1. After transfer, wash nitrocellulose membrane with 25 mL TBS for 5 min at room temperature.

Step 2. Incubate membrane in 25 mL of blocking buffer for 1 hr at room temperature.

Step 3. Wash three times for 5 min each with 15 mL of TBST.

Step 4. Incubate membrane and primary antibody (at the appropriate dilution and diluent as recommended in the product datasheet) in 10 mL primary antibody dilution buffer with gentle agitation overnight at 4° C.

Step 5. Wash three times for 5 min each with 15 mL of TBST.

Step 6. Incubate membrane with the secondary antibody in 10 mL of blocking buffer with gentle agitation for 1 hr at room temperature.

Step 7. Wash three times for 5 min each with 15 mL of TBST.

### Protein Detection:

Step 1. Incubate membrane with 10 mL SignalFire™ (5 mL Reagent A, 5 mL Reagent B) with gentle agitation for 1 min at room temperature.

Step 2. Drain membrane of excess developing solution (do not let dry), wrap in plastic wrap and expose to x-ray film. An initial 10-sec exposure should indicate the proper exposure time.

To evaluate the expression of MMP2 in vivo, mice were injected with A375 SM-Luc cells. After 6 weeks, the lungs were harvested, fixed and stained following standard IHC protocols using a monoclonal MMP2 antibody and a peroxidase-labeled secondary antibody. The following immunohistochemistry protocol was utilized:

A. Deparaffinization/Rehydration:

Step 1. Deparaffinize/hydrate sections:
a. Incubate sections in three washes of xylene for 5 min each.
b. Incubate sections in two washes of 100% ethanol for 10 min each.
c. Incubate sections in two washes of 95% ethanol for 10 min each.

Step 2. Wash sections two times in dH₂O for 5 min each.

B. Antigen Unmasking:

Step 1. For Citrate: Bring slides to a boil in 10 mM sodium citrate buffer, pH 6.0; maintain at a sub-boiling temperature for 10 min. Cool slides on bench top for 30 min.

Step 2. For EDTA: Bring slides to a boil in 1 mM EDTA, pH 8.0: follow with 15 min at a sub-boiling temperature. No cooling is necessary.

Step 3. For TE: Bring slides to a boil in 10 mM Tris/1 mM EDTA, pH 9.0: then maintain at a sub-boiling temperature for 18 min. Cool at room temperature for 30 min.

Step 4. For Pepsin: Digest for 10 min at 37° C.

C. Staining:

Step 1. Wash sections in dH₂O three times for 5 min each.

Step 2. Incubate sections in 3% hydrogen peroxide for 10 min.

Step 3. Wash sections in dH₂O two times for 5 min each.

Step 4. Wash sections in wash buffer for 5 min.

Step 5. Block each section with 100-400 μL blocking solution for 1 hr at room temperature.

Step 6. Remove blocking solution and add 100-400 μL primary antibody diluted in recommended antibody diluent to each section*. Incubate overnight at 4° C.

Step 7. Equilibrate SignalStain® Boost Detection Reagent to room temperature.

Step 8. Remove antibody solution and wash sections with wash buffer three times for 5 min each.

Step 9. Cover section with 1-3 drops SignalStain® Boost Detection Reagent as needed. Incubate in a humidified chamber for 30 min at room temperature.

Step 10. Wash sections three times with wash buffer for 5 min each.

Step 11. Add 1 drop (30 μL) SignalStain® DAB Chromogen Concentrate to 1 mL Signal Stain® DAB Diluent and mix well before use.

Step 12. Apply 100-400 μL SignalStain® DAB to each section and monitor closely. 1-10 min generally provides an acceptable staining intensity.

Step 13. Immerse slides in dH₂O.

Step 14. If desired, counterstain sections with hematoxylin per manufacturer's instructions.

Step 15. Wash sections in dH₂O two times for 5 min each.

Step 16. Dehydrate sections:

a. Incubate sections in 95% ethanol two times for 10 sec each.

b. Repeat in 100% ethanol, incubating sections two times for 10 sec each.

c. Repeat in xylene, incubating sections two times for 10 sec each.

Step 17. Mount sections with coverslips.

Random images were captured with an optical microscope.

Accumulation of ESMSV in Cancer Cells in Vivo. To confirm the percentage of fluorescent particles in cancer cells and in lungs, flow cytometry (Fortessa, BD FACS, San Jose, Calif., USA) was used to analyze the live cells ex vivo. Briefly, mice with melanoma lung metastasis (6 weeks after injection of melanoma cells) were sacrificed 24 hr after injection of PLGA-PEG NPs, PLGA-PEG NPs-conjugated silicon microparticles, or the MMP2 substrate-modified, PLGA-PEG NPs-conjugated silicon microparticles with the same amount of coumarin 6. Lungs with human A375 melanoma tumor nodules were minced and digested in DMEM/F12 medium containing 300 U/mL collagenase, 100 mg tissue/mL medium for 1.5 hr at 37° C. The resultant suspension was sequentially resuspended and filtrated through a 40-μm mesh to obtain single-cell suspension. For flow cytometry analysis, dead cells were isolated with CYTOX® Blue (Life Technologies), and human A375 melanoma cells were stained with APC-Cy7 Anti-human HLA-ABC antibody (BD Bioscience, San Jose, Calif., USA).

Results and Discussion

Design, Synthesis, and Characterization of ESMSV. The design and synthesis process of ESMSV is shown in FIG. 1A and FIG. 1B. A MMP2 substrate peptide having the amino acid sequence AGFSGPLGMWSAGSFG (SEQ ID NO:1) was used as a linker for conjugation of PLGA-PEG nanoparticles and mesoporous silicon microparticles. Coumarin 6, considered a model hydrophobic cargo, was encapsulated into the PLGA nanoparticles. Meanwhile, the negative AF555 siRNA, considered as model siRNA, was encapsulated into the DOPC liposomes. The DOPC liposomes were loaded in the pores of the silicon microparticles to show that the silicon microparticles could transport nanoparticles both in pores and on its surface. In this example, conjugated polymeric nanoparticles on the surface were able to stimulate release through the cleavage of MMP2 enzyme.

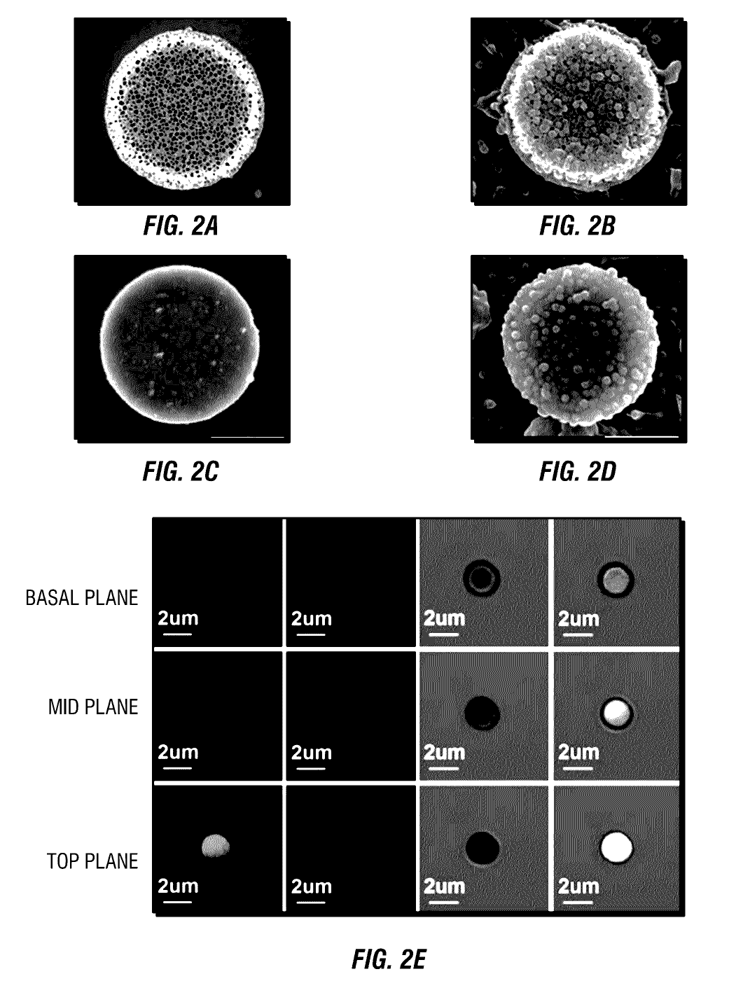

The morphology of mesoporous silicon microparticles and ESMSV are shown in FIG. 2A, FIG. 2B, FIG. 2C, and FIG. 2D. It was mesoporous on the front of the silicon microparticles, while there was a thin solid base on the back. After the conjugation, both the front and back of ESMSV showed homogenously conjugated PLGA NPs. The fluorescent signal from the confocal microscope confirmed that the AF555 siRNA-loaded liposomes were mainly distributed inside the pores and on the top plane of silicon microparticles, while the coumarin 6-loaded PLGA NPs were mainly conjugated to the top and bottom surface of the microparticles (FIG. 2E).

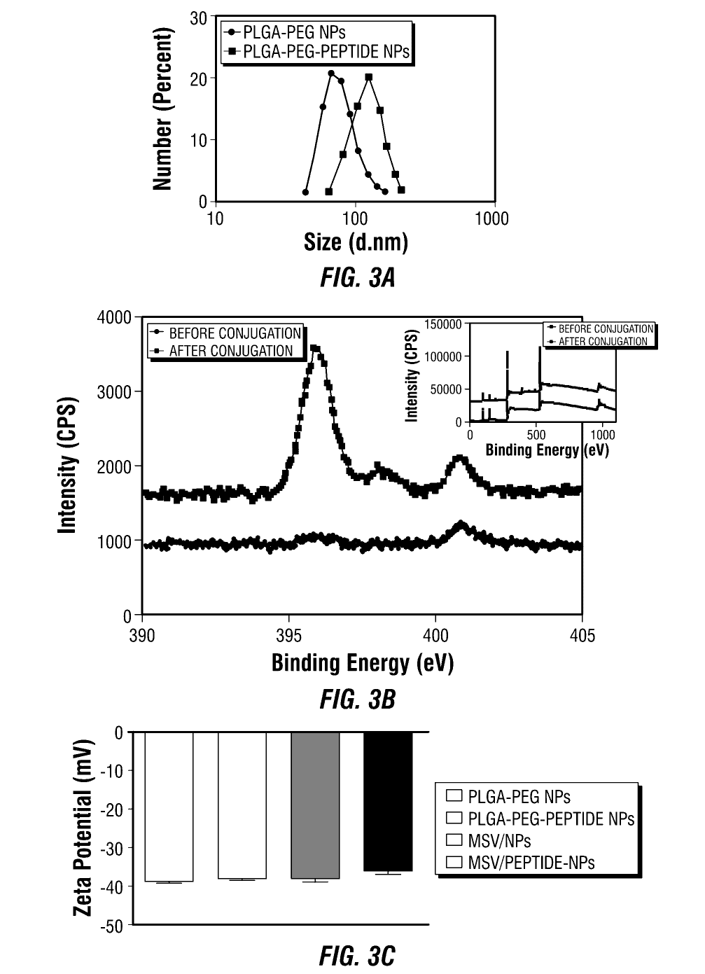

The size distribution of PLGA-PEG NPs and PLGA-PEG-Peptide (MMP2 substrate) was measured by the dynamic light scattering (DLS) (FIG. 3A). Both of them showed narrow distribution around 100 nm. There was an ˜20 nm increase of PLGA NPs in size after conjugation of the peptide. The conjugation could be further confirmed by the surface chemistry of PLGA NPs before and after conjugation of the peptide using X-ray photoelectron spectroscopy (XPS) (FIG. 3B). The signal at the position corresponding to the binding energy of nitrogen is (N 1 s) was compared before and after conjugation. A nitrogen signal could be detected from the MMP2 substrate, but not from the PLGA-PEG-COOH NPs. The appearance of the nitrogen signal indicated successful conjugation of the peptide to the surface of the PLGA NPs.

Surface charge of the particles was reflected by zeta potential. The zeta potentials of PLGA NPs, PLGA-PEG-Peptide NPs and their conjugation with silicon microparticles, nominated as MSV/NPs and MSV/Peptide-NPs, were measured to be approximately −40 mV with limited increase following conjugation. The relatively high zeta potential guaranteed sufficient colloidal stability, and less toxicity for normal cells than positively-charged particles (FIG. 3C).

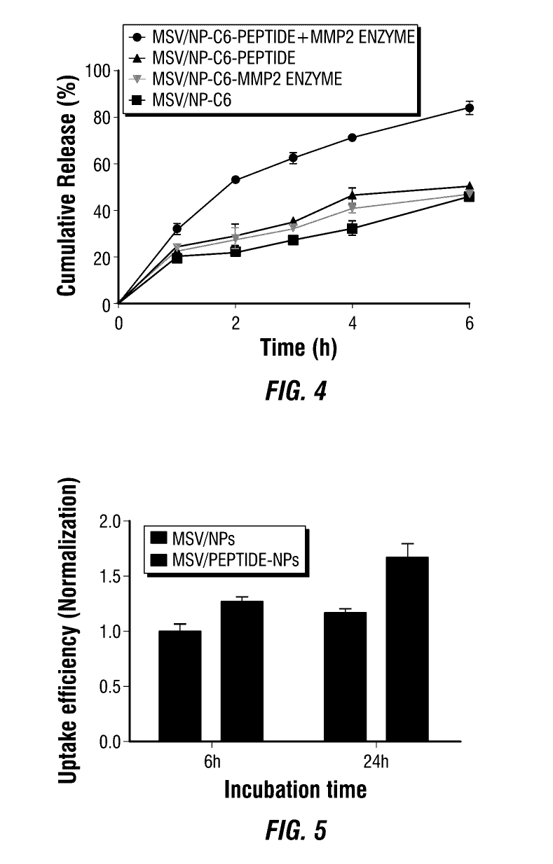

Stimuli Release of ESMSV In Vitro. The enzyme-stimuli release of the PLGA NPs from the silicon microparticles were demonstrated in vitro by comparing the fluorescent intensity in the release buffer (50 mM HEPES containing 10 mM CaCl₂) of MSV/NPs and MSV/Peptide NPs with or without MMP2 enzymes at designated times (FIG. 4). Through controlling the centrifuge speed, only the micro/nano particles were able to be spun down, while the released PLGA NPs as well as the released fluorescent dyes could be detected in the release buffer. It showed that without the cleavage of MMP2 enzymes, only about 40% of the coumarin 6 was released within 6 hrs; whereas in the cleavage of MMP2 enzymes, about 80% of the coumarin 6 was released. The significant increase could be only achieved by the release of PLGA NPs from the silicon microparticles through triggering of MMP2 enzymes (The PLGA NPs were conjugated to the MSV by MMP2 substrate peptide. MMP2 enzyme is able to cut that peptide. When there is MMPs enzyme, PLGA NPs can be cleaved from the MSVs).

Enhanced Cellular Uptake of ESMSV. The enzyme-stimuli release of PLGA NPs improved the cellular uptake efficiency. The cellular uptake efficiency of PLGA NPs conjugated silicon microparticles (MSV/NPs) and PLGA-Peptide (MMP2 substrate) NPs (MSV/Peptide NPs) conjugated silicon microparticles were compared (FIG. 5) by measuring the fluorescent intensity in A375 human melanoma cells after incubation with the coumarin 6-loaded particles with the emergence of MMP2 enzyme in the medium. The result showed that the cellular uptake of stimuli-release particles increased to 1.2-fold after 6-hrs' incubation and to 1.5-fold after 24-hrs' incubation, as compared to the non-stimuli-release particles. The improvement was caused by the difference of cellular uptake efficiency between nanoparticles and microparticles. It is well known that nanoparticles of ˜100 nm diameter exhibit a higher cellular uptake efficiency than microparticles due to the balance of binding energy and transportation efficiency during the process of endocytosis. The cleavage of PLGA NPs from silicon microparticles by MMP2 enzymes assisted with MSV/Peptide NPs to transport more therapeutics into the cells. The nanoparticles showed higher cellular uptake efficiency than the microparticles. As the micro/nano composite changes the biodistribution of the nanoparticles, the stimuli release of NPs enhances the cellular uptake at the accumulated organs if there is over-expressed MMP2 enzyme in vivo. Microparticles accumulate more at lungs than nanoparticles, while nanoparticles show higher cellular uptake efficiency. Because nanoparticles can be stimuli-released at the target tumor site using the disclosed ESMSV, the drug delivery systems possess the advantages of both particle types.

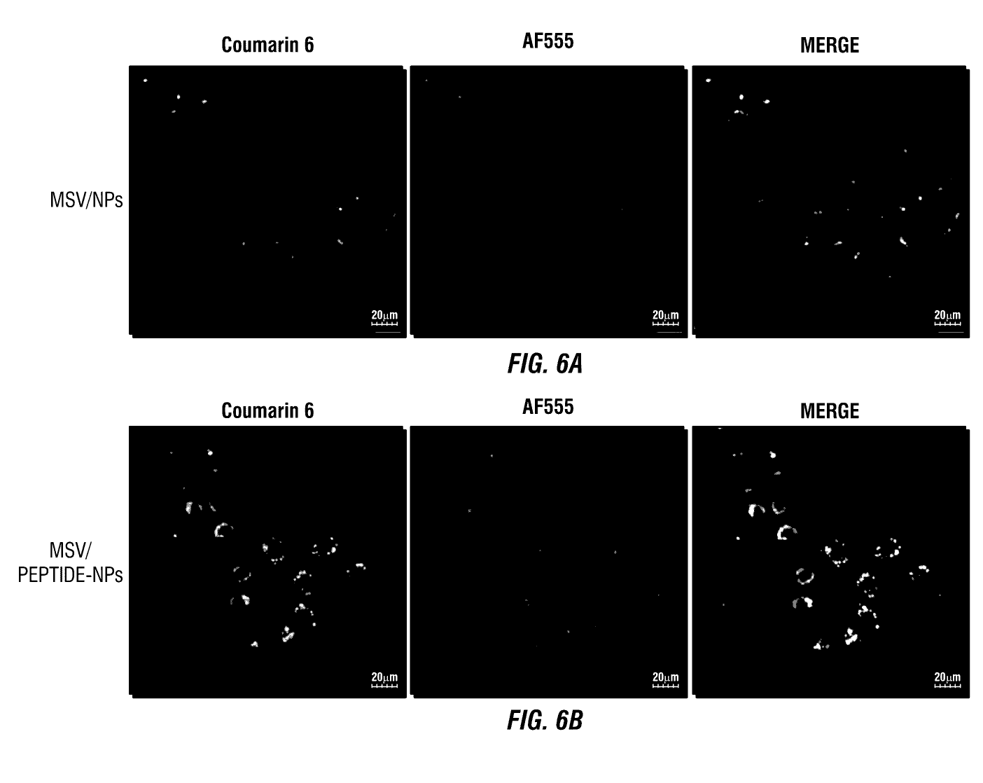

The cellular internalization of ESMSV and non-enzyme-stimulated MSV was further confirmed visually on A375 melanoma cells after 24 hr by confocal microscopy (FIG. 6A and FIG. 6B). The green dot showed the uptake of the MSV, while the dispersed green fluorescence showed the uptake of PLGA NPs. With the help of MMP2 enzyme, the total cellular uptake of coumarin 6-loaded PLGA NPs on the surface of ESMSV increased significantly through internalization of both micro- and nano-particles. A slight increase was also observed for siRNA-loaded DOPC liposomes inside the pores of ESMSV.

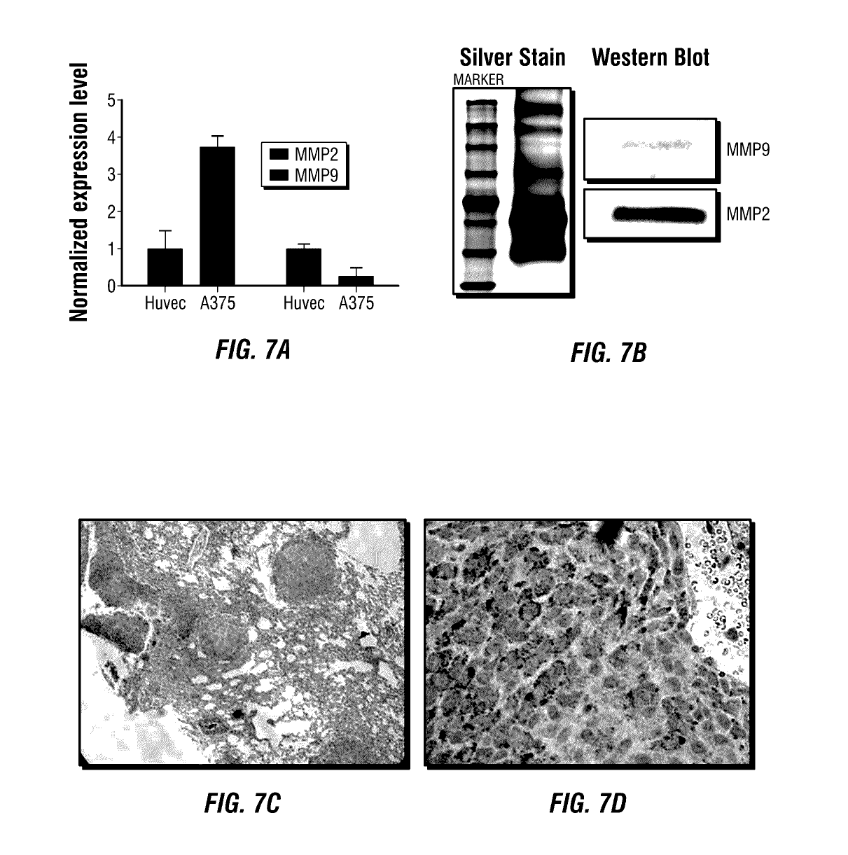

Expression and Excretion of MMP2 in a Melanoma Lung Metastasis Mice ModeL Next, the expression and excretion of MMP2 enzyme was demonstrated in a melanoma lung metastasis model, proving the facility of the ESMSV in vivo. First, the mRNA expression of MMP2 and MMP9 enzyme was confirmed by RT-PCR in A375 melanoma cells. It showed that MMP2 enzyme was overexpressed in A375 cells compared with that in HUVEC cells as well as the expression of MMP9 in both cells (FIG. 7A). Next, the excretion of MMP2 enzyme was confirmed in vitro in the cell culture medium without FBS. Both the silver staining and western blot showed that A375 was able to excrete MMP2 in vitro (FIG. 7B). The excretion of MMP2 enzyme was then demonstrated in the A375 melanoma lung metastasis mice model. The section of lung tissues with A375 metastasis was stained by immunohistochemistry. A great amount of MMP2 enzymes were detected around A375 tumor cells (FIG. 7C and FIG. 7D).

Enhanced Transportation of Therapeutics in Tumor Lung Metastasis. A further flow cytometry study demonstrated that ESMSV were able to enhance transportation of therapeutics in the metastatic tumor cells in lung.

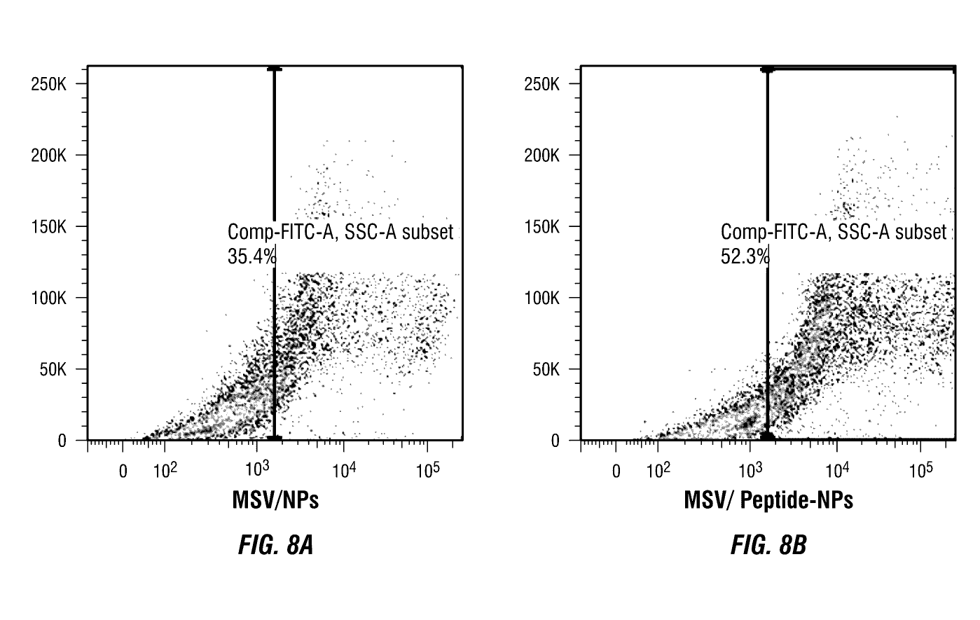

The mice treated with coumarin 6-loaded PLGA-PEG NPs, MSV/NPs or the ESMSV were sacrificed 24 hr after the injection. The lungs were extracted and digested to collect all the cells and they were then marked with HLA antibody. The cells were sorted by flow cytometry as lung cells (HLA-negative) and A375 melanoma cells (HLA-positive) (FIG. 8A and FIG. 8B). For the coumarin 6 delivered by the PLGA-PEG NPs, 1.38% population of the lung cells presented coumarin 6-positive fluorescent signal and 21.2% population of melanoma cells presented coumarin 6-positive signal. For the coumarin 6 delivered by the MSV/NPs, 3.46% population of the lung cells presented positive signal and 40.1% population of melanoma cells presented positive signal, a 1.5-fold and 0.9-fold increase, respectively. The results proved that the composite increased the lung accumulation, lung retention time, and the delivery efficiency in lung metastatic cancer cells.

### Example 2

### A Micro/Nano Composite for Combination Therapy of Melanoma Lung Metastasis

Nanoparticles, around 10 nm to 200 nm, show advantages in cellular uptake and EPR effect. However, they show rapid clearance by MPS in liver or spleen, as well as short retention time in lungs. This example describes a micro/nano composite (MNC) that increases accumulation of nanoparticles in the lung, and which provides a new combinational therapy of melanoma lung metastasis. This new composite, which is considered as a multistage delivery system, is composed of mesoporous silicon microdisks with siRNA-loaded liposomes inside its pores and docetaxel-loaded PLGA-PEG nanoparticles conjugated to its surface. Compared with liposomes or PLGA-PEG nanoparticles, the composite shows increased accumulation both in lung tissues and metastatic melanoma cells in lungs. This example demonstrates the synergistic anti-tumor effect of the MNC in vitro and in vivo using a mouse model bearing melanoma lung metastasis. The therapeutic effect is better than that of nanoparticles without being loaded on the silicon microdisks, or that of the silicon microdisks with monotherapy nanoparticles. Besides, without the silicon microdisks, the mixture of liposomes and nanoparticles cannot achieve better efficacy than the respective monotherapy. The result provides a novel strategy to improve current therapeutic effect of cancer lung metastasis by nanoparticles, which is utilizing the multistage MNC to improve lung accumulation and to achieve combination therapy.

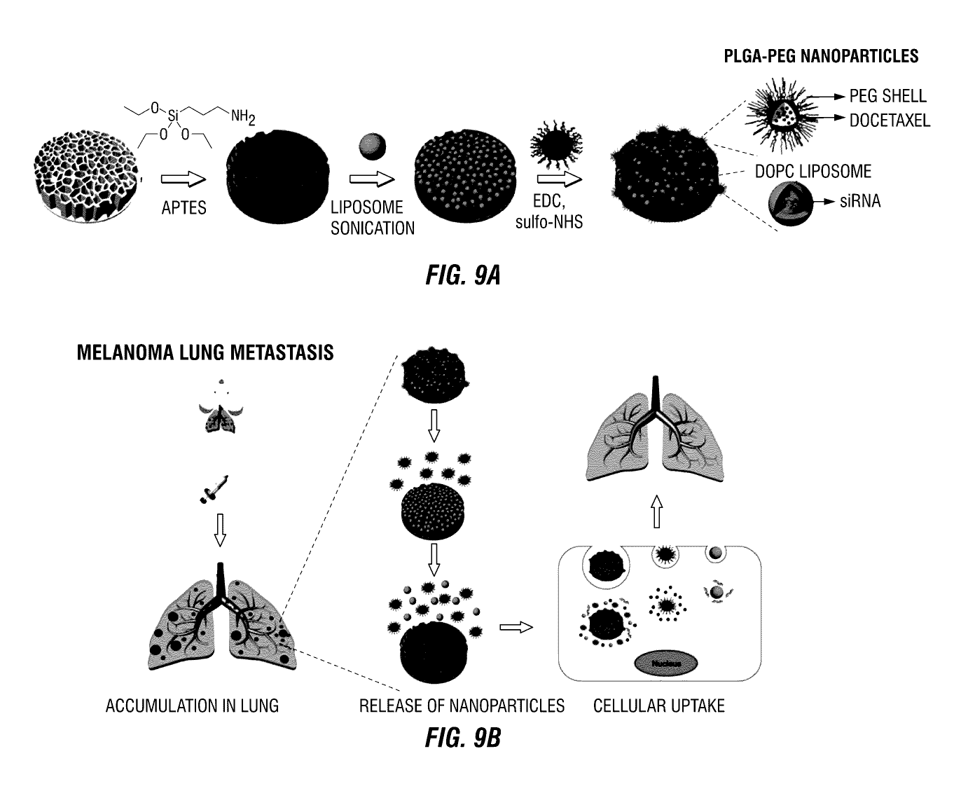

The MNC improved lung accumulation and retention time of nanoparticles for therapy of cancer lung metastasis (FIG. 9A and FIG. 9B). The strategy was to conjugate the PLGA-PEG nanoparticles (NPs) around 100 nm to the surface of mesoporous silicon microdisks around 2.6 μm in diameter and 0.7 μm in height. After the conjugation, a cookie-like structure is fabricated with PLGA-PEG NPs homodispersed on the surface of microdisks. The PEG increases the conjugation probability between the nano- and micro-particles, forms a shell to prolong the circulation time of the composite. At the same time, small nanoparticles, such as liposomes or micelles, can be loaded inside the pores before conjugation. The strategy makes it possible to transport nanoparticles with size range from 10-200 nm by microdisks. The full use of the pores and surface of the microdisks improves the loading efficiency and loading stability of the composite. The composite will have good property of transportation in blood. More importantly, they will improve the lung accumulation of the nanoparticles by remaining in the abundant and small capillary (as small as 1 μm) of lungs. The nanoparticles delivered by the microdisks are released by the biodegradation of the silicon microdisks during the retention in the lungs. These nanoparticles can then transport therapeutics into the cancer cells on the lungs by EPR effect and endocytosis. The composite combines the advantages of nanoparticles and microdisks. The prolonged retention time of the composite in lungs will help to deliver more therapeutics in the lesions of lungs.

The therapeutic effect of the composite was evaluated on the mice bearing melanoma lung metastasis (Li et al., 1989; Craft, 2005; Xu et al., 2008). Melanoma is the most serious type of skin cancer with high mortality (Jang and Atkins, 2013). It shows high metastasis in lung, liver, bone, and brain. Among all organs, lung is the most common metastatic site with the highest likelihood around 70-80%. The most commonly mutant gene in metastatic melanoma is BRAF V600E mutation (Davies et al., 2002; Kumar et al., 2003; Salama and Flaherty, 2013; Ribas and Flaherty, 2011). The mutation leads to reduced survival. The inhibition of mutant BRAF leads to decreased VEGF secretion, reduced vascularity, and tumor apoptosis (Flaherty et al., 2010). Currently, a problem in melanoma therapy is the resistance to BRAF inhibitors (Garber, 2013). For example, the response rate for trametinib is 22%, and that for vemurafenib, dabrafenib, or LGX818 is approximately 40%-60% (Flaherty et al., 2012; McArthur et al., 2014). Studies reveal that the resistant mechanisms include secondary mutations in BRAF V600E, reactivation of MAPK and activation of alternative pathway. One solution is to combine different therapeutic agents to overcome the resistance (Tran et al., 2008; Coffee et al., 2013; Smalley and Flaherty, 2009; Shannon et al., 2009). For example, combination of Braf and Mek inhibitors were approved by FDA last year. There are also ongoing clinical trials with docetaxel and Braf inhibitor (Gupta et al., 2014).

In the present disclosure, micro/nano composites have been developed as drug delivery agents for combination therapy of cancers such as human melanoma lung metastasis. In exemplary embodiments, the composite included a duotherapy comprising BRAF-specific siRNA and the chemotherapeutic agent, docetaxel. The transfection efficiency and synergistic delivery effects were evaluated in vitro. Accumulation in lung tissue and efficacy of the anti-cancer compound was evaluated in vivo, and compared to the same therapeutics delivered either by liposomes or nanoparticles (without the silicon microdisks), or by the composite containing only a single (monotherapeutic) chemotherapeutic agent. The disclosed micro/nano composite delivery agents disclosed herein represent an efficient platform for combinational therapies and provide new strategies for improving traditional nanoparticles for use in the treatment of cancers such as melanoma lung metastasis.

Materials and Methods

Materials. PLGA-PEG-COOH was synthesized according to a previous publication (Cheng et al., 2007). PLGA (50:50, carboxylate end group, inherent viscosity 0.20 dL/g) was purchased from Lactel Absorbable Polymers (Pelham, Ala., USA). NH₂-PEG-COOH (MW 2000) was purchased from Laysan Bio, Inc. (Arab, Ala., USA). Docetaxel (>99%) was purchased from LC Laboratories (Woburn, Mass., USA). Sulfo-NHS was purchased from Thermo-Fisher Scientific, Inc. DiR dye was purchased from Life Technologies. DOPC was purchased from Avanti Polar Lipids, Inc. (Alabaster, Ala., USA). Allstar Neg siRNA specific for either AF555 or AF647 were purchased from Qiagen (Germantown, Md., USA). Fetal bovine serum (FBS) was purchased from Atlas Biologicals (Fort Collins, Colo., USA). Luciferin potassium salt was purchased from Gold Biotechnology, Inc. (St. Louis, Mo., USA). All antibodies were purchased from Cell Signaling Technology (Danvers, Mass., USA). Cell Counting Kit-8 was purchased from Dojindo Molecular Technologies, Inc. (Santa Clara, Calif., USA). PBS, DMEM, trypsin, penicillin, streptomycin, and all anti-BRAF siRNAs were purchased from GE Healthcare Life Sciences (Pittsburgh, Pa., USA). All the other chemicals were purchased from Sigma-Aldrich (St. Louis, Mo., USA).

Preparation and Characterization of MNC. The mesoporous silicon microdisks with the size of 2.6 μm in diameter, 0.7 μm in height and ˜50 to 60 nm average pore diameter were fabricated by photolithography and electrochemical etching. The microdisks were modified with 3-aminopropyltriethoxysilane (APTES) according to a previous report (Chiappini et al., 2010). Docetaxel-loaded PLGA-PEG nanoparticles were prepared by nanoprecipitation as described previously (Kamaly et al., 2013). In the brief, weighed amounts of PLGA-PEG-COOH and docetaxel with a ratio of 20:1 was dissolved in acetone with a polymer concentration of 5 mg/mL. The solution was added drop wise into ultrapure water with an oil-to-water ratio of 1:2 under vigorous stirring. After 6 hr, the suspension was washed and centrifuged twice at 2000 rpm and 12,000 rpm for 20 min at 4° C. The same procedure was applied to synthesize the fluorescent coumarin-6- or DiR-loaded PLGA-PEG nanoparticles with docetaxel replaced by 1 wt % coumarin-6 or DiR. The siRNA-loaded DOPC liposomes were prepared according to published methods (Fine et al., 2013). In brief, the corresponding siRNA (anti-BRAF, Neg. siRNA AF555 or AF647, 1 mg/mL in H₂O), DOPC (20 mg/mL in t-butanol), Tween-20® (1.2% vol./vol. in H₂O) and t-butanol mix with a volume ratio of 1:0.5:0.5:42 under vortex. The mixture was freeze-dried under vacuum, and then rehydrated with 1× PBS buffer at 0° C. for a few minutes under sonication.

To prepare the composite, 1 billion APTES-modified silicon microdisks were washed twice with pure water. siRNA-loaded liposomes were hydrated and mixed with the microdisks. The mixture was sonicated at 0° C. for several minutes. After sonication, the liposomes-loaded microdisks were washed and centrifuged with PBS twice at 4500 rpm for further use. The PLGA-PEG nanoparticles, synthesized from 200 mg PLGA-PEG with carboxyl group on the surface, were activated by EDC (60 mg) and sulfo-NHS (48 mg) in PBS for 30 min, and then were added into the solution of liposomes-loaded microdisks. The final volume of the solution was 20 mL and the reaction lasted for 3 hr. After the reaction, the micro/nano composite was washed and centrifuged 3 times with water at 2000 rpm.

The size and zeta potential of the particles were measured by the Zetasizer Nano (Malvern Instruments). The SEM characterization was shown on an ultra-high resolution scanning electron microscope (SEM 230, NovaNano, Hillsboro, Oreg., USA). Western blots were performed using standard protocols on 4%-15% Bio-Rad Mini-Protean TGX Precast Gels (Bio-Rad, Hercules, Calif., USA).

Controlled Release Profile. The MNC with docetaxel-loaded PLGA-PEG nanoparticles and AF555 siRNA-loaded liposomes were dispersed in buffers with different pH values (pH=5.0, pH=7.4) containing 0.1% (vol./vol.) Tween-80®, which improved the solubility of docetaxel. The dispersion in tubes was then incubated in an orbital shaker water bath (120 rpm, 37° C.). At designated time intervals, the sample tubes were centrifuged at 10,000 rpm for 20 min. The pellet was drained and resuspended in fresh buffer to continue the drug release process. The supernatant was separated into two parts. One was extracted by DCM and transferred in the mobile phase (50% acetonitrile in water in volume ratio). The solution was then filtered by 0.45-mm PVDF membrane for HPLC analysis. The column effluent was analyzed at 230 nm by HPLC (Hitachi, Dallas, Tex., USA) for the amount of released docetaxel. The other part was added into black 96-well microplates and the fluorescence intensity was recorded (Genios, Tecan, Mannedorf, Switzerland) for the amount of released AF555-specific siRNA. The error bars were obtained from triplicate samples.

Confocal Microscopy. After reaching confluence, cells were detached, counted, and seeded in a 4-well cover glass chamber (LAB-TEK, Nagle-Nunc, Ill., USA) overnight. Then, the medium was replaced by the composite with coumarin 6-loaded PLGA-PEG nanoparticles and AF555-specific siRNA-loaded liposomes in cell culture medium at concentration of 0.1 billion/mL and incubated for different periods. The cells were washed twice with pre-warmed 1× PBS and fixed with 70% ethanol for 20 min. After that, the cells were washed twice with PBS and then the nuclei were counterstained by DAPI for 45 min. The cells were washed again twice by 1× PBS and immersed in 1× PBS for confocal microscopic imaging.

Cell Culture. A375 human melanoma cells were obtained from ATCC. Cells were cultured in DMEM with 10% (vol./vol.) fetal bovine serum (FBS) and 1% (vol./vol.) penicillin/streptomycin in an incubator at 37° C. under 5% CO₂. The cells were sub-cultivated at 70% confluence with 0.25% trypsin.

In Vitro Cytotoxicity. A375 cells were seeded in 96-well transparent plates (Costar) at 3×10³ cells/well (0.1 mL). After 12 hr, the medium was replaced by the suspension of the composite with designated concentrations. The composites were sterilized with UV irradiation overnight before the experiment. After 72 hr, cell viability was measured using the following standard protocol for assaying CCK-8:

Step 1. Dispense 100 μL of cell suspension (5000 cells/well) in a 96-well plate. Pre-incubate the plate for 24 hrs in a humidified incubator at 37° C., 5% CO₂.

Step 2. Add 10 μL of various concentrations of substances to be tested to the plate.

Step 3. Incubate the plate for an appropriate length of time (e.g., 6, 12, 24, or 48 hours) in the incubator.

Step 4. Add 10 μL of CCK-8 solution to each well of the plate. Be careful not to introduce bubbles to the wells, since they interfere with the O.D. reading.

Step 5. Incubate the plate for 1-4 hrs in the incubator.

Step 6. Measure the absorbance at 450 nm using a microplate reader.

Animal Model. The animal studies were performed in accordance with the guidelines of the Animal Welfare Act and the Guide for the Care and Use of Laboratory Animals following protocols approved by the Institutional Animal Care and Use Committee (IACUC). Six-week-old female athymic nude mice were purchased from Charles River Laboratories (Boston, Mass., USA). A375 SM-Luc human melanoma cancer cells with stable expression of luciferase were harvested from exponential cultures and intravenously inoculated in the tail vein with 10⁶ cells per mouse. The lung metastasis was monitored using a bioluminescence imaging system (IVIS 200).

Biodistribution. Mice with melanoma lung metastasis (6 weeks after injection of melanoma cells) were injected intravenously with different fluorescent particles for analysis of biodistribution. Biodistribution in tissues (heart, liver, spleen, lung, and kidney) was measured 6-and 24-hrs after injection with 1 mg/kg AF647 siRNA loaded in the liposomes, or 0.2 mg/kg DiR dye loaded in the PLGA-PEG nanoparticles or the composite with the same amount of the fluorescent agents. Fluorescence of AF647 or DiR dye was quantified and normalized by individual tissue weight.

To confirm the percentage of fluorescent particles in cancer cells and in lungs, flow cytometry (Fortessa, BD FACS; San Jose, Calif., USA) was used to analyze the live cells ex vivo. Briefly, mice with melanoma lung metastasis (6 weeks after injection of melanoma cells) were sacrificed 24 hr after injection of 1 mg/kg AF555 siRNA loaded in the liposomes, or 0.2 mg/kg coumarin 6-loaded in the PLGA-PEG nanoparticles, or the composite with the same amount of the fluorescent agents. Lungs with human A375 melanoma tumor nodules were minced and digested in DMEM/F12 medium containing 300 U/mL collagenase (100 mg/mL) for 1.5 hr at 37° C. The resultant suspension was sequentially resuspended and filtrated through a 40-μm mesh to obtain single-cell suspension. For flow cytometry analysis, dead cells were isolated with CYTOX® Blue (Life Technologies) and human A375 melanoma cells were stained with APC-Cy7 anti-human HLA-ABC antibody (BD Bioscience). The following general protocol was employed:

Step 1. Aliquot 50 μL of cell suspension to each tube or well.

Step 2. Combine the recommended quantity of each primary antibody in an appropriate volume of Flow Cytometry Staining Buffer (eFluor® NC Flow Cytometry Staining Buffer) so that the final staining volume is 100 μL (i.e., 50 μL of cell sample+50 μL of antibody mix) and add to cells. Pulse vortex gently to mix.

Step 3. Incubate 30 min in the dark on ice or at 4° C.

Step 4. Wash the cells by adding Flow Cytometry Staining Buffer (eFluor® NC Flow Cytometry Staining Buffer). Use 2 mL for tubes or 200 μL/well for microtiter plates. Pellet the cells by centrifugation at 300-400×g at 4° C. for 5 min. Repeat for a total of two washes, discarding supernatant between washes.

Step 5. Resuspend stained cells in Flow Cytometry Staining Buffer (eFluor® NC Flow Cytometry Staining Buffer).

Step 6. Add a viability dye PI (5 μL/sample) to each sample to exclude dead cells from analysis.

Step 7. Acquire data on a flow cytometer.

In Vivo Anti-Cancer Efficacy. The mice bearing melanoma lung metastasis (two weeks after injection of melanoma cells) were monitored by intraperitoneal injection of luciferin and by quantifying the bioluminescence signal from lungs (IVIS imaging). The mice were divided into different groups randomly. From day 0, the mice were intravenously injected with the composite (with 0.6 mg/kg BRAF-specific siRNA and 4 mg/kg docetaxel), or other particles with the same amount of therapeutic agents, dispersed in PBS and PBS only as negative control group every 7 days for 4 times. The mice were quantified by the bioluminescent signal from lungs every 7 days (IVIS imaging). At Day 38, some of the mice were sacrificed for counting nodules, taking photos of lungs and hematoxylin and eosin staining. For the immunohistochemistry (IHC) staining, the mice bearing melanoma lung metastasis (six weeks after injection of melanoma cells) were intravenously injected with different particles. The mice were sacrificed and lungs were extracted 72 hr after injection of the particles. The lungs were then fixed and stained under standard IHC staining protocol with BRAF-specific antibody. The images within random area of nodules on lungs were observed by optical microscopy.

Confocal Microscopy. The micro/nano composite (MNC) containing coumarin 6-loaded PLGA-PEG nanoparticles and AF555-labeled siRNA-loaded liposomes was suspended in water, dropped on a 4-well cover glass chamber, and sealed for confocal microscopy imaging.

In Vitro Anticancer Activity. A375 cells and A375SM luciferase expressing (A375SM-Luc) cells were seeded in 96-well plates (Costar, INVENTORS: CITY? XXXXXXXX IL, USA) at a density of 3×10³ cells/well (0.1 mL). After 12 hrs, cells were exposed to treatment groups and cell viability was measured after 72 hrs using the Cell Counting Kit-8 (CCK-8) (Dojindo Molecular Technologies, Inc.).

Animal Weight Measurements. Treatment of mice bearing A375SM melanoma lung metastases was initiated two weeks after cancer cell injection. Mice received weekly intravenous injections of particles (BRAF siRNA: 1 mg/kg; docetaxel: 4 mg/kg) for four weeks. Animal weights were recorded once a week.

DNA Sequence Analysis. The genomic DNA of A377 cells, A375-SM cells, and A375-SM-Luc cells was isolated using the QIAamp® DNA Mini Kit (Qiagen) according to the manufacturer's instructions. Polymerase chain reactions (PCR) were performed to confirm the presence of the BRAF V600E mutation.

The following primers were used for the reaction:

(SEQ ID NO: 2)
5′-GCATCTCACCTCATCCTAACAC-3′
and
(SEQ ID NO: 3)
5′-CTAGTAACTCAGCAGCATCTCA-3′.

Amplification was performed as follows: 95° C. for 10 min, 30 cycles of 94° C. for 15 sec, 58° C. for 30 sec, 72° C. for 30 sec, and 72° C. for 5 min. The PCR products were purified with MinElute® PCR purification kit (Qiagen), and then sequenced using the following primer:

(SEQ ID NO: 4)
5′-GCATCTCACCTCATCCTAACAC-3′.

Results

Design, Synthesis, and Characterization of the Composite. The composite is synthesized step by step. It is composed of three parts: the silicon microdisks, the siRNA-loaded liposomes, and the docetaxel-loaded PLGA-PEG nanoparticles (FIG. 9A). The silicon microdisks with the size of 2.6 μm in diameter, 0.7 μm in height and 50˜60 nm in pores were fabricated first by photolithography and electrochemical etching from silicon wafers. They were modified by APTES for further conjugation. Then, the BRAF siRNA-loaded DOPC liposomes were prepared and loaded into the pores of the modified microdisks by sonication to form liposomes. Docetaxel-loaded PLGA-PEG nanoparticles were synthesized by nanoprecipitation. Here PEG₂₀₀₀ was used to increase the conjugation probability as well as to avoid rapid clearance of the composite by macrophage. These PLGA-PEG NPs were then conjugated to the surface of the silicon microdisks. The composite is considered as a multistage delivery system (multistage vector, MSV). The silicon microdisks are considered as the first stage vector to deliver the liposomes and nanoparticles to the lungs (FIG. 9B). Due to the shape and size effect, the first stage will help to increase the accumulation of the particles in lungs and increase the retention time there. Liposomes and nanoparticles, considered as the second stage vectors, are loaded into the silicon microdisks by both physical adsorption inside the pores and chemical conjugation on the surface. Such strategy enables the silicon microdisks to deliver a variety of particles with a wide size range. The second stage vectors fulfilled with therapeutic agents will release in the lungs by biodegradation of the first stage vectors. The nano-sized particles will then penetrate into the cancer cells by endocytosis. Compared with nanoparticles, the micro/nano composite is believed to be more efficient for lung accumulation and lung cancer therapy.

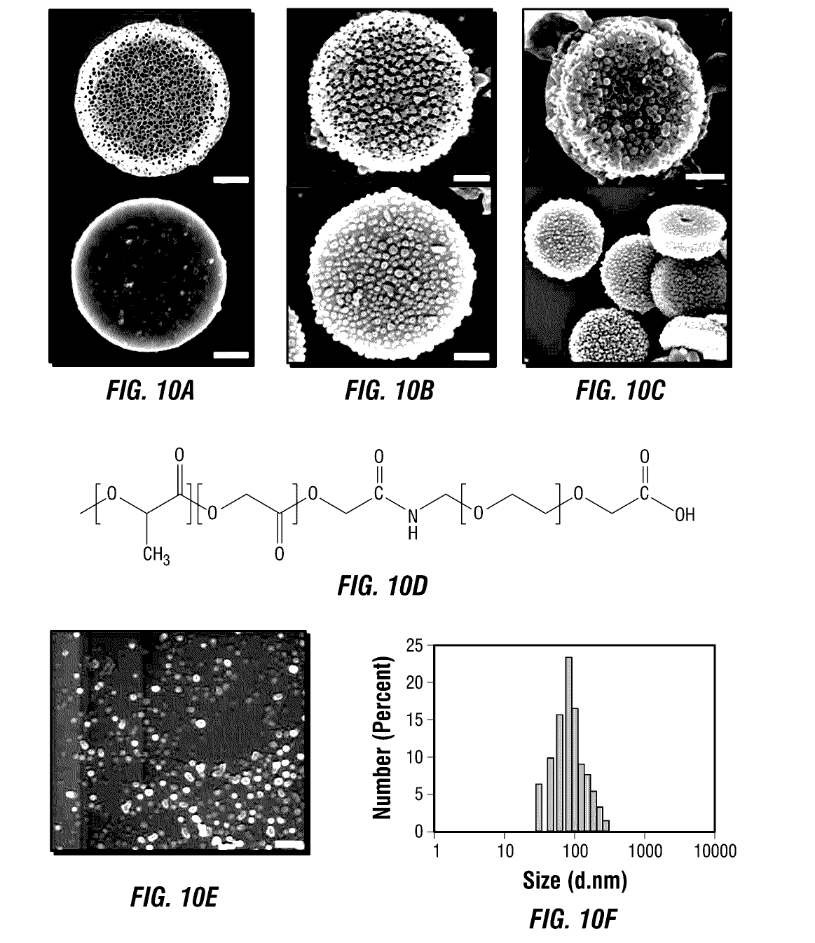

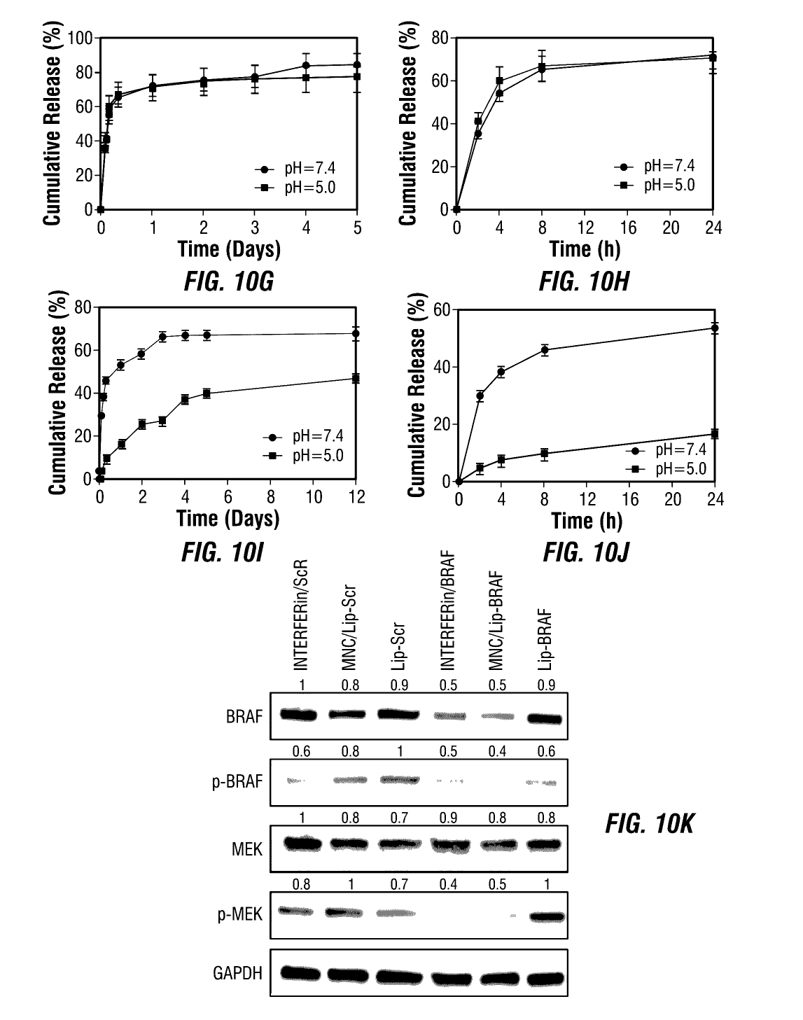

The morphology of the composite is shown in FIG. 10A, FIG. 10B, and FIG. 10C. After loading and conjugation of nanoparticles, the silicon microdisks present a cookies-like appearance with uniform PLGA-PEG nanoparticles on their surface. The characterization of silicon microdisks, PLGA-PEG NPs, and the composite is shown in FIG. 10D, FIG. 10E, and FIG. 10F and Table 1.

TABLE 1
CHARACTERIZATION OF EXEMPLARY MICRO/NANO COMPOSITES (MNCS)

| | | Zeta | | | |
|---|---|---|---|---|---|
| | | Potential | Docetaxel | | |
| | Size | (mV) | Loading | | siRNA Loading |
| Silicon | 2.6 μm * 0.7 μm | 25.3 ± 0.9 | N/A | | N/A |
| microdisk | | | | | |
| PLGA-PEG | 87.48 ± 1.82 nm | −30.3 ± 0.5 | 52.61 ± 0.52 | μg/mg | N/A |
| nanoparticle | | | | | |
| MNC/Lip-Braf | 2.6 μm * 0.7 μm | −38.8 ± 0.9 | 803.55 ± 53.50 | μg/billion | 114.90 ± 11.76 μg/billion |
| &NP- | | | | | |
| docetaxel | | | | | |

Lip, liposomes;
NP, polymeric nanoparticle;
PEG, polyethylene glycol;
PLGA, poly(lactic-co-glycolic acid).

The composite showed a highly negative surface charge (38.8 eV), and achieved a docetaxel loading of ˜804 μg/billion composites and an siRNA loading of ˜105 μg/billion composites.

The release profile of the composite is evaluated in vitro in two different buffers of pH=5.0 and pH=7.4. There was no significant difference of release for docetaxel at pH=5.0 or pH=7.4 (FIG. 10G and FIG. 10H). The composite showed a burst release of 70% docetaxel in the first 24 hr and a moderate release of the remaining 15% from day 1 to day 5. The release profile showed advantages in killing the massive tumor cells efficiently at the beginning and then keeping the therapeutic effect for long period. It indicates the release mechanism as diffusion-controlled release. Meanwhile, when evaluating the release of siRNA from the composite, the pH value affects the rate dramatically (FIG. 10I and FIG. 10J). In the first 24 hr, there is ˜16% siRNA released at pH=5.0, whereas ˜50% siRNA released when pH=7.4. From then on to 12^{th} days, ˜50% siRNA was released when pH=5.0 and ˜70% siRNA was released when pH=7.4. The reason of pH-related release of siRNA is due to the accelerated degradation of the silicon microdisks in alkaline environment (FIG. 10K). This indicated that the mechanism of siRNA release from the composite was a degradation-controlled release.

To confirm the efficiency of siRNA delivery by the composite, the expression of BRAF, phosphor-BRAF, and its downstream MEK and phosphor-MEK was investigated in A375 melanoma cells after treatment with commercial transfection method (INTERFERin®, Polypus Transfection, Illkirch, FRANCE), siRNA-loaded DOPC liposomes and siRNA-loaded composite for 72 hr. The expression of BRAF, phosphor-BRAF, and phosphor-MEK decreased 37.5%, 50%, and 50%, respectively, as efficient as the commercial one.

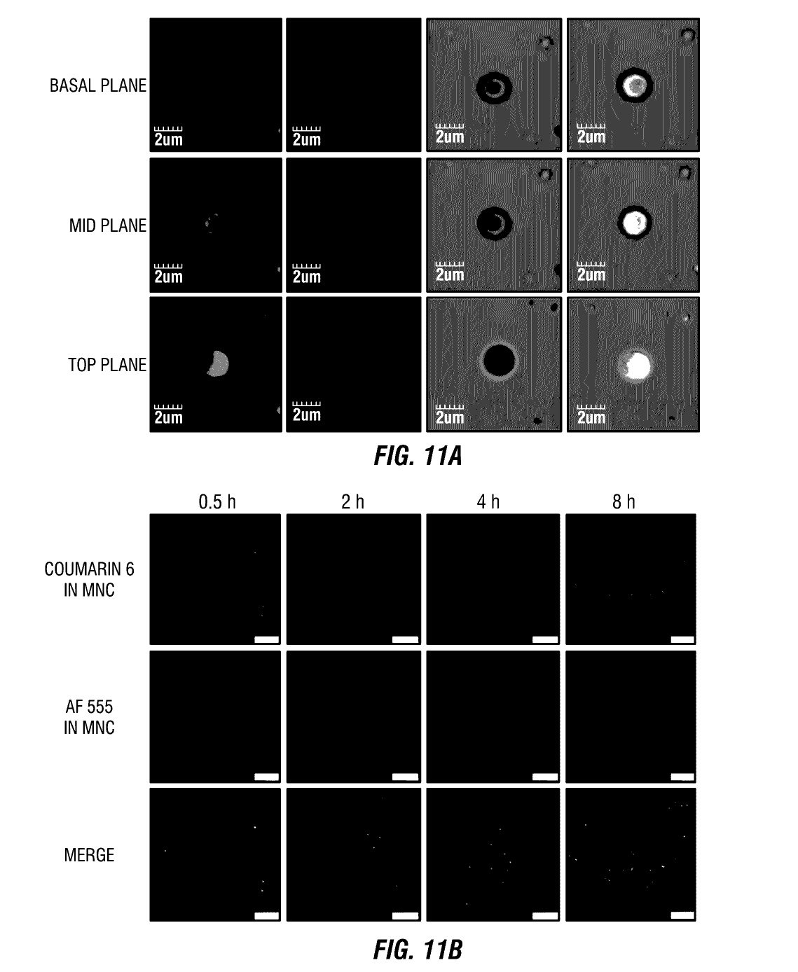

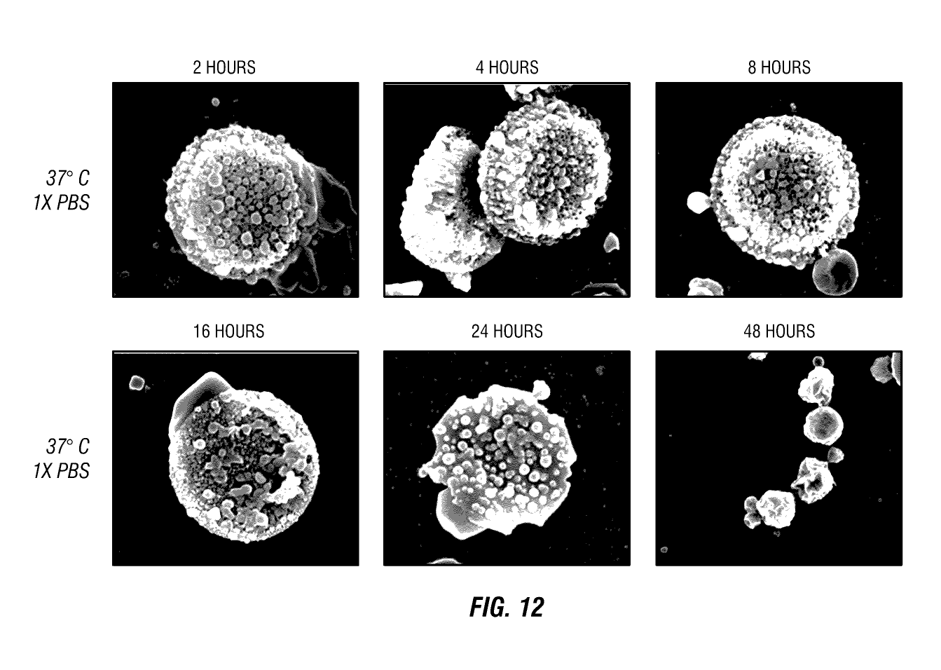

The co-localization of siRNA and chemotherapeutics was examined by the composite in vitro with confocal microscopy. FIG. 11A shows different focused planes of the composite. The green fluorescence reveals that the coumarin 6-loaded PLGA-PEG NPs distribute on the top surface and side surface of the silicon microdisks. The red fluorescence shows that the AF555 siRNA-loaded liposomes distribute both in pores and on surfaces of the microdisks. The distribution of the loading agents on the silicon microdisks was further confirmed by a standing particle in FIG. 12. Then, the co-localized delivery of the composite was evaluated in A375 cells (FIG. 11B). The results showed continued cellular uptake of the composite with prolonged incubation time. The merged yellow fluorescence around cell nuclei (blue fluorescence) demonstrated the co-localized delivery of siRNA and chemotherapeutics by the composite in vitro.

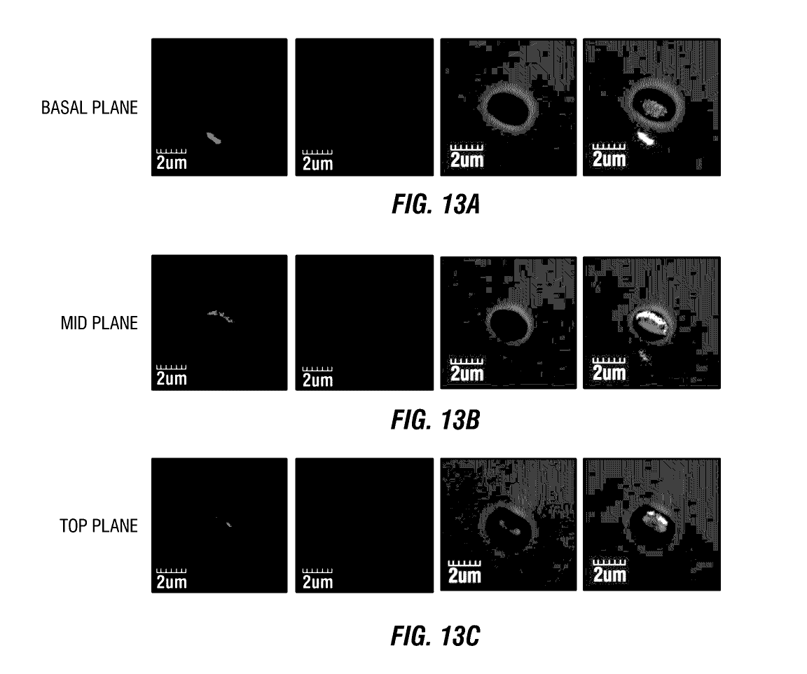

In Vitro Synergistic Anti-Cancer Effect of the Composite. The therapeutic effect of the composite was first investigated in vitro on A375 melanoma cells. The therapeutic effect of BRAF siRNA was checked with different sequences (FIG. 13A, FIG. 13B, and FIG. 13C, Table 2).

TABLE 2
EXEMPLARY BRAF V600E siRNA SEQUENCES

| Name | siRNA Sequence |
|---|---|
| BRAF V600E siRNA 1 | 5′-GCUACAGAGAAAUCUCGAU-3′ |
| (selected) | (SEQ ID NO: 5) |
| BRAF V600E siRNA 2 | 5′-AACAGUCUACAAGGGAAAGUG-3′ |
| | (SEQ ID NO: 6) |
| BRAF V600E siRNA 3 | 5′-GCUACAGAGAAAUCUCGAU-3′ |
| | (SEQ ID NO: 7) |

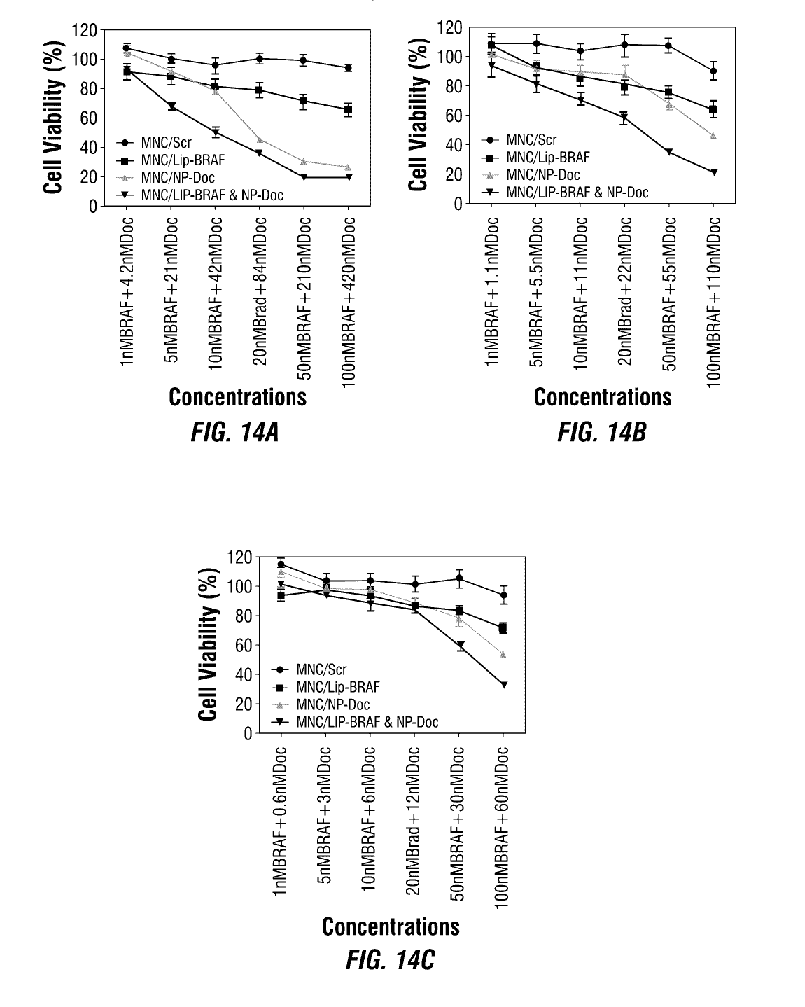

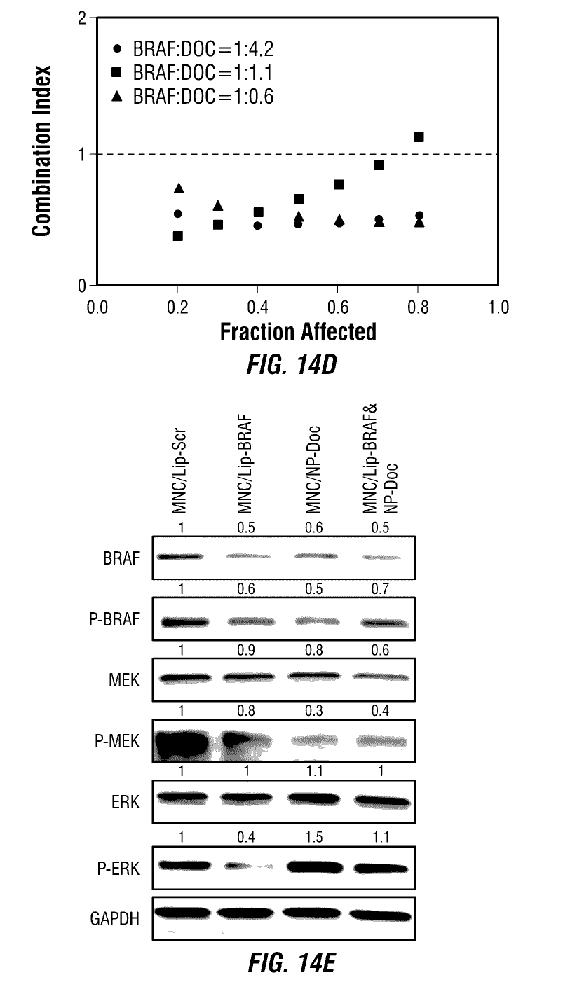

The cell viability was evaluated after treatment by the composite with different siRNA and docetaxel ratios: 1:4.2, 1:1.1, and 1:0.6 (FIG. 14A, FIG. 14B, and FIG. 14C, respectively). The IC₅₀'s are shown in Table 3:

TABLE 3
HALF-MAXIMAL INHIBITORY CONCENTRATION
(IC₅₀) VALUES FOR MNCs LOADED
WITH VARIOUS BRAF SIRNA-TO-DOCETAXEL RATIOS

| | Microdisk/NP- | Microdisk/Lip- | Microdisk/Lip-BRAF & |
|---|---|---|---|
| IC₅₀ | Docetaxel | BRAF | NP-Docetaxel |
| 1:4.2 | 128.1 nM docetaxel | N/A | 12.9 nM siRNA + |
| | | | 54.3 nM Docetaxel |
| 1:1.1 | 68.0 nM docetaxel | N/A | 27.0 nM siRNA + |
| | | | 29.7 nM Docetaxel |
| 1:0.6 | 50.2 nM docetaxel | N/A | 48.2 nM siRNA + |
| | | | 28.9 nM Docetaxel |

These data demonstrated that when dose ratio of BRAF siRNA and docetaxel is different, the composite leads to different cell growth inhibitory abilities. By adjusting the ratio of the loaded liposomes and the conjugated PLGA-PEG NPs, the composite could easily and precisely control the dose ratio. Compared with sequence administration of docetaxel and siRNA, simultaneous delivery by the composite controllable and optimum dose ratio could be achieved at the lesions for an improved therapeutic outcome. By determining the combination index (FIG. 14D), it was shown that the composite delivering both siRNA and docetaxel with dose ratio of 1:4.2 and 1:0.6 resulted in synergistic anti-cancer effect. There combination index is around 0.4˜0.6. A dosage ratio of 1:1.1 showed synergistic effect with the combination index around 0.4 at high growth inhibitory rate, but no synergistic effect at low growth inhibitory rate.

To understand the possible mechanism for synergism of the composite, the protein expression of BRAF pathway was analyzed, including BRAF, phosphor BRAF, MEK, phosphor-MEK, ERK, and phosphor-ERK treated with the composite, or the composite with only BRAF siRNA-loaded liposomes, or only docetaxel-loaded PLGA-PEG NPs (FIG. 14E). As BRAF is an important oncogene in melanoma, its knockdown leads to cell death. The result showed that BRAF siRNA functioned to knockdown BRAF expression and its related phosphor-proteins downstream. While for docetaxel, it also decreased the expression of BRAF, phosphor-BRAF and phosphor-MEK, but increased the expression of phosphor-ERK.

Many studies have reported that docetaxel can activate ERK and phosphor-ERK, which is considered as one of its resistances. When combining BRAF siRNA and docetaxel by the composite, BRAF siRNA helps to decrease the expression of phosphor-ERK activated by docetaxel, leading to attenuate drug resistance. Meanwhile, docetaxel helps to enhance the knockdown of MEK and phosphor-MEK led by BRAF siRNA, leading to better cell inhibitory effect. The interaction with the BRAF pathway is considered as a possible mechanism for the synergistic anti-cancer effect observed in A375 melanoma cells.

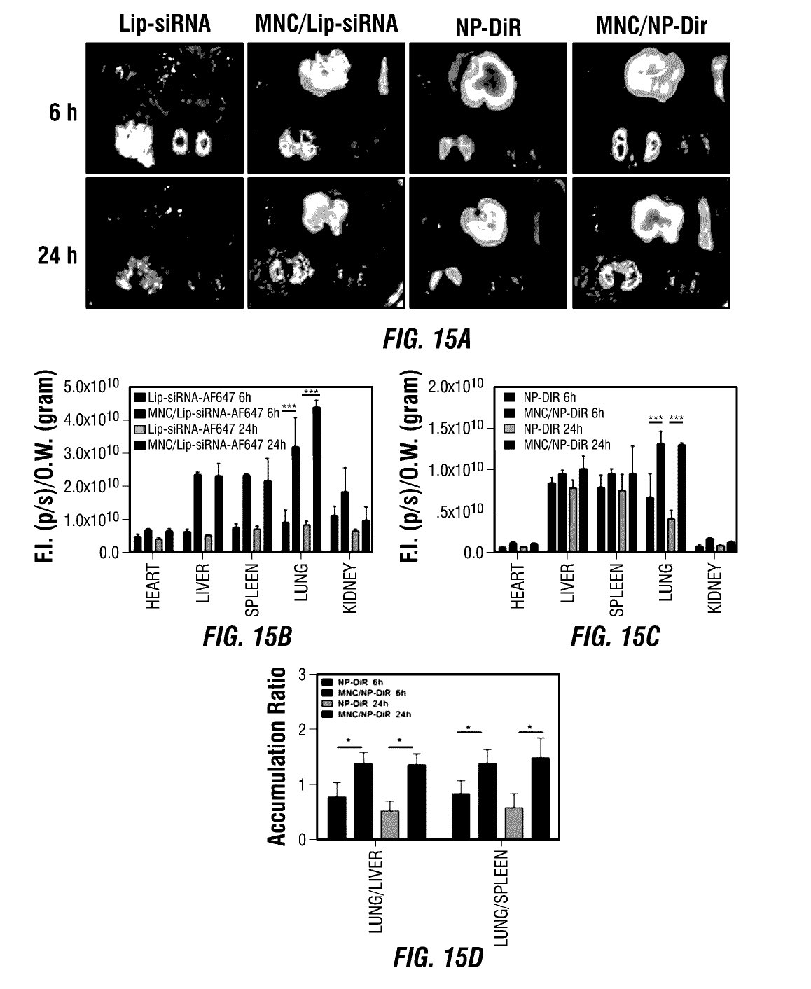

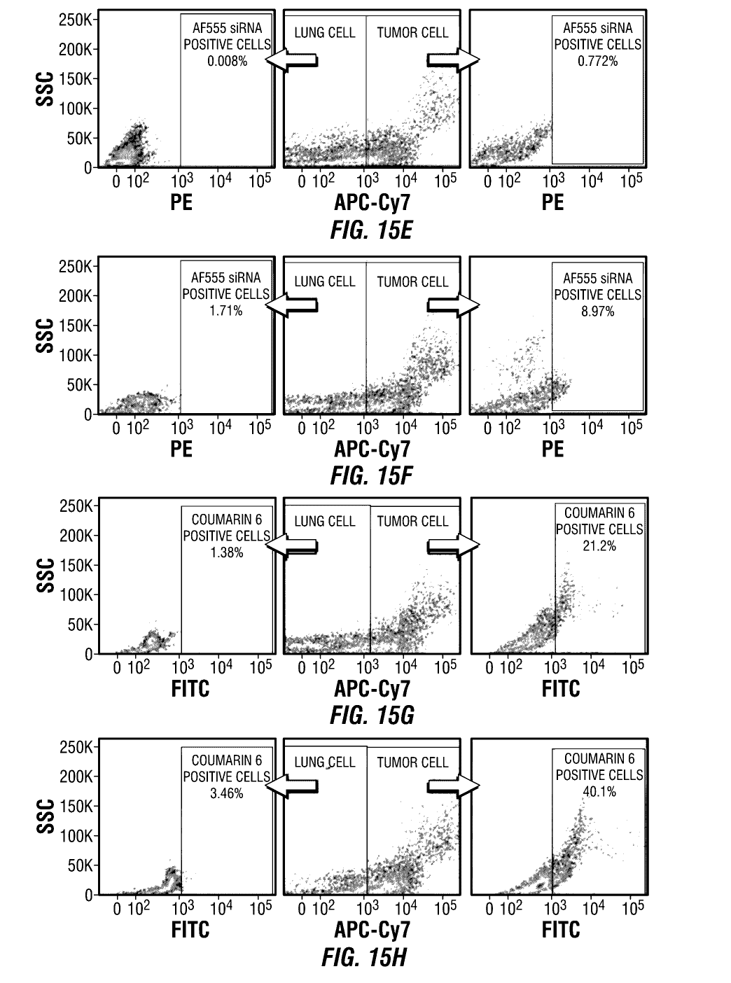

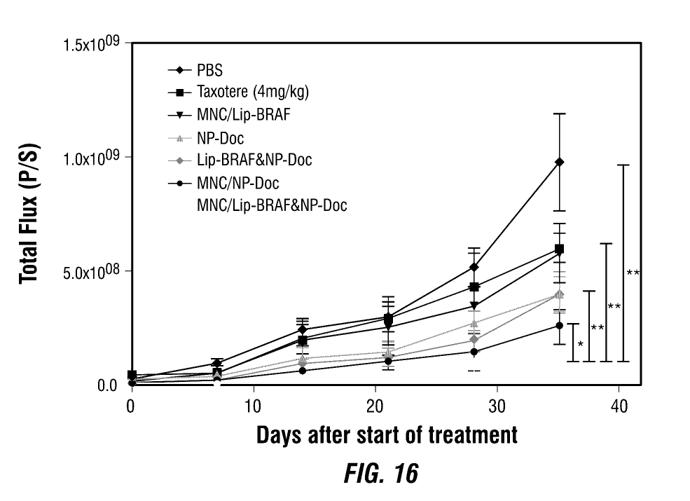

Lung Accumulation of the Composite. The biodistribution of AF647-tagged siRNA-loaded liposomes, DiR-loaded PLGA-PEG nanoparticles, and the composite loaded with the same fluorescent dyes were evaluated after intravenous injection into mice bearing the melanoma lung metastasis. Fluorescence of the particles was examined in heart, liver, spleen, lung, and kidneys ex vivo. FIG. 15A shows that after 6 hr, most of the fluorescent liposomes accumulated in the kidneys, and they were cleared after 24 hr. While for the composite, it transported the fluorescent cargo to the liver and lungs after 6 hr. They were kept in the lungs and appeared the most accumulation compared with other organs after 24 hr (other two groups are shown in FIG. 16). Quantitative analysis showed that the siRNA amount in the lungs delivered by the composite was 3.6-fold and 5.5-fold more than that delivered by liposomes 6 hr and 24 hr after injection, respectively (FIG. 15B).

Next, the biodistribution of PLGA-PEG NPs and the composite was examined. In FIG. 15A, most of the DiR-loaded PLGA-PEG NPs accumulated in the liver after 6 hr. The fluorescent signal decayed after 24 hr indicating the clearance of the NPs. After conjugating the NPs to the silicon microdisks, as the composite, the most accumulated organs changed from the liver to the lungs and after 24 hr the signal didn't decrease obviously. Quantitative analysis showed that compared with PLGA-PEG NPs, the lung accumulation of the fluorescent signal delivered by the composite increased 2-fold and 3.2-fold at 6 hr and 24 hr after injection, respectively (FIG. 15C). The accumulation ratio of lung-to-liver and lung-to-spleen was determined. For the PLGA-PEG NPs, the ratio of lung-to-liver was 0.78 for 6 hr and 0.53 for 24 hr; ratio of lung-to-spleen was 0.84 for 6 hr and 0.58 for 24 hr. While for the composite, the ratio of lung to liver was 1.39 for 6 hr and 1.37 for 24 hr, increasing 78% and 158%, respectively; the ratio of lung to spleen was 1.39 for 6 hr and 1.49 for 24 hr, increasing 65% and 157%, respectively (FIG. 15D).

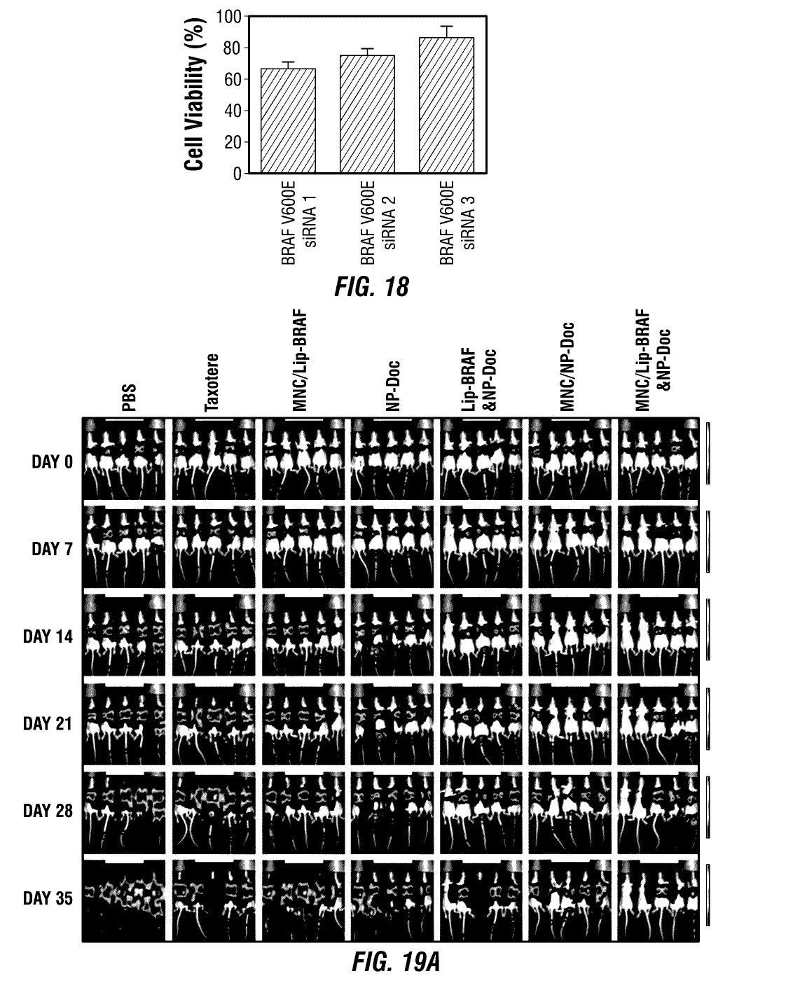

The above experiment demonstrated the increased lung accumulation by the composite. Next, it was shown that the accumulated composite transported the therapeutics not only to the lungs but also to the nodules on the lungs. It can be achieved by biodegradation of the silicon microdisks and release of the nanoparticles. First, the biodegradation rate of the composite in vitro in PBS buffer was analyzed, and it was found that the silicon microdisks could be degraded after 48 hr (FIG. 17). The cellular uptake efficiency of PLGA-PEG nanoparticles and the micro/nano composite were compared, and the superior cellular uptake efficiency of the nanoparticles was shown (FIG. 18). Next, the nodule accumulation of the therapeutics was demonstrated by flow cytometry. The mice treated with AF555-tagged siRNA-loaded liposomes, coumarin 6-loaded PLGA-PEG NPs, or the composite with the same dyes, were sacrificed 24 hr after injection. The lungs were extracted and digested, and the cells were collected and marked with HLA antibody. The cells were sorted by the flow cytometer as lung cells (HLA negative) and A375 melanoma cells (HLA positive) (FIG. 15E, FIG. 15F, FIG. 15G, and FIG. 15H). Further analysis showed that for the AF555-tagged siRNA delivered by the liposomes, 0.008% population of the lung cells presented positive fluorescent signal and 0.772% population of melanoma cells presented positive signal. For the AF555-tagged siRNA delivered by the composite, 1.71% population of the lung cells presented positive signal and 8.97% population of melanoma cells presented positive signal. There was a 10-fold increase of accumulation in melanoma cells after delivered by the composite. For the coumarin 6 delivered by the PLGA-PEG NPs, 1.38% population of the lung cells presented positive fluorescent signal and 21.2% population of melanoma cells presented positive signal. For the coumarin 6 delivered by the composite, 3.46% population of the lung cells presented positive signal and 40.1% population of melanoma cells presented positive signal, a 1.5-fold and 0.9-fold increase, respectively. The results prove that the composite increases the lung accumulation, lung retention time as well as the delivery efficiency in lung metastatic cancer cells.

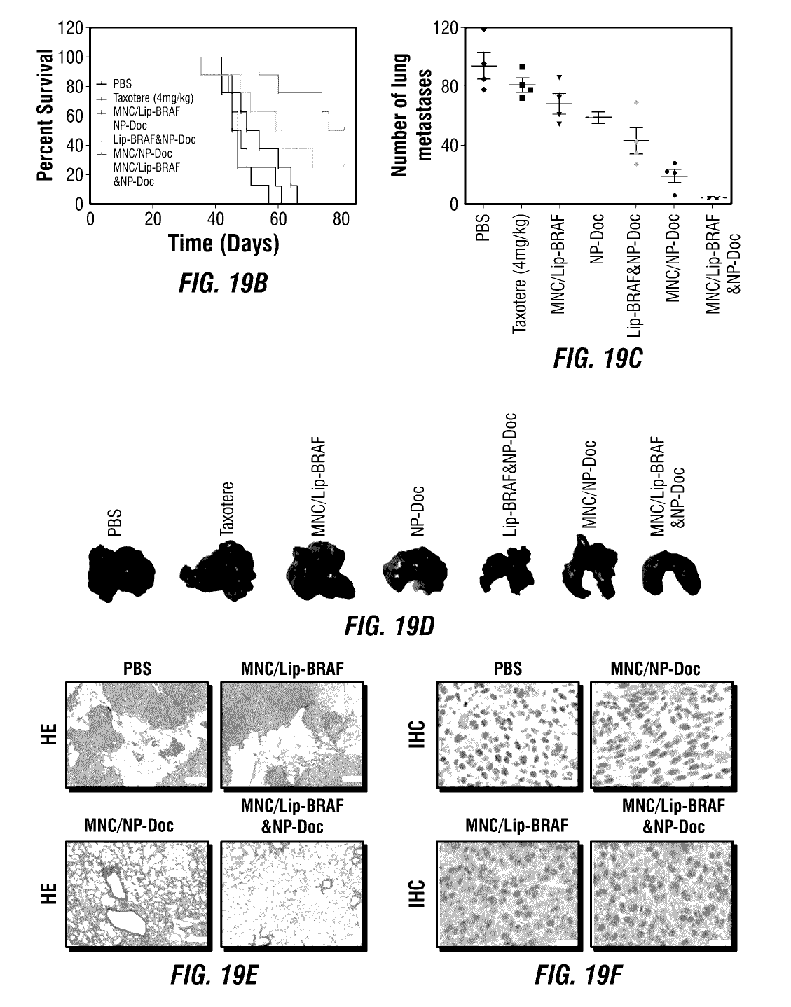

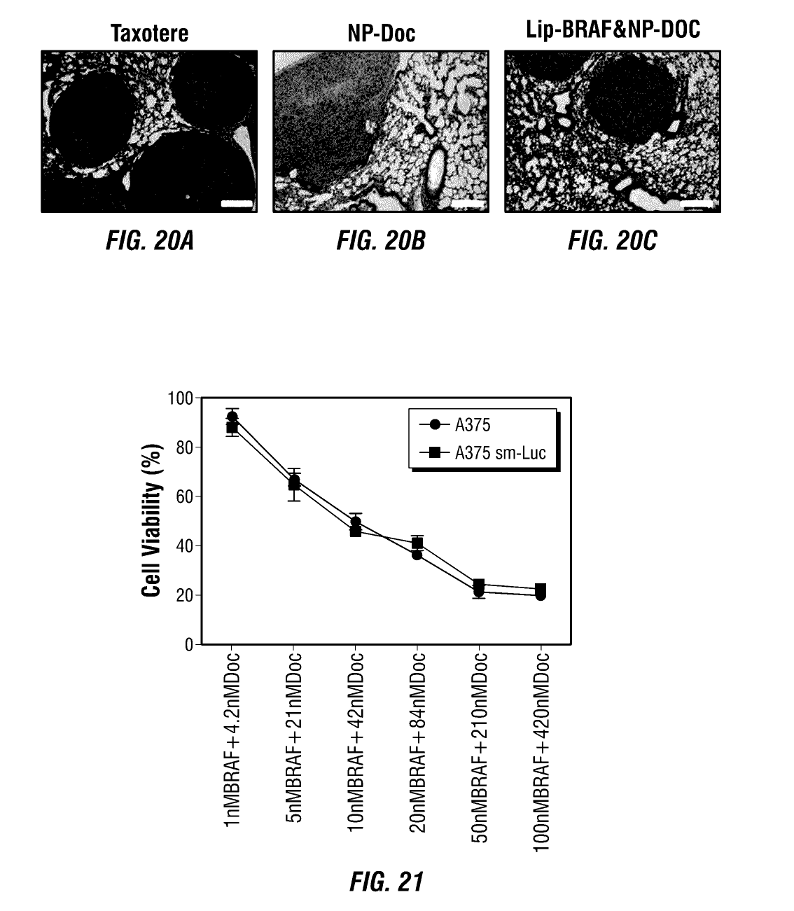

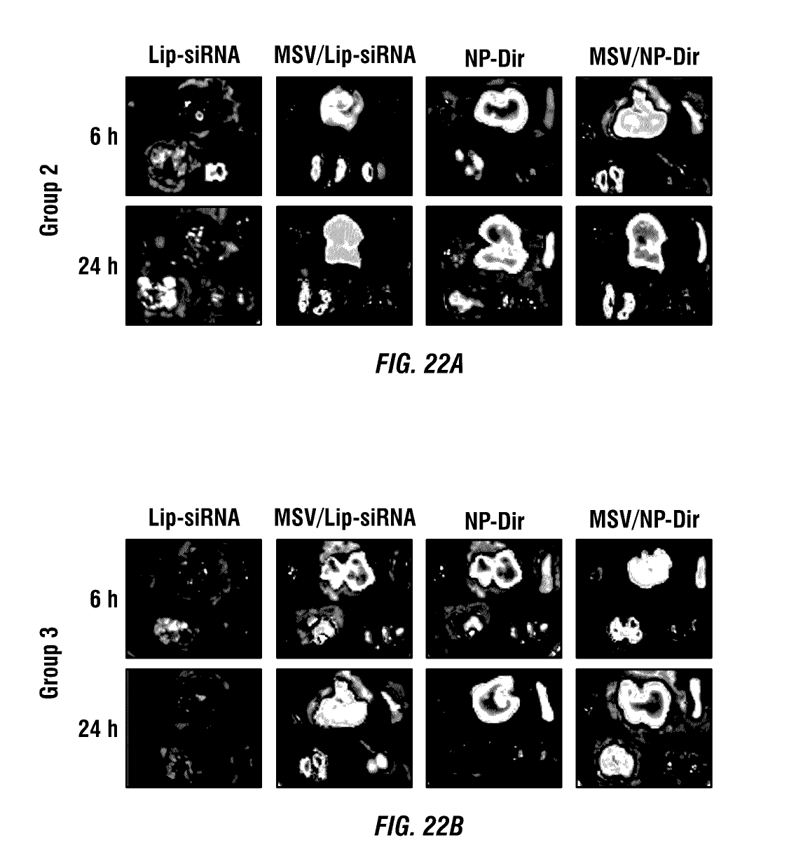

In Vivo Anti-Cancer Effect of the Composite. The therapeutic effect of the composite was estimated and compared with nanoparticles and monotherapy on the mice bearing melanoma lung metastasis. The therapeutic effect was monitored by analyzing the fluorescence signal from luciferase-labeled A375 melanoma cells in vivo (FIG. 19A). Quantitative data is shown in FIG. 20A, FIG. 20B, and FIG. 20C. These super lung metastatic A375 (A375 sm-luc) melanoma cells with luciferase expression were first compared with the normal A375 cells. DNA sequence analysis showed that the BRAF V600E mutant of both cells (FIG. 21). Cytotoxicity test on A375 and A375 sm-luc showed that they had similar response to the composite (FIG. 22A and FIG. 22B). After 35 days of treatment, compared with the negative PBS group, different formulations of docetaxel presented different effects on inhibition of tumor growth. The silicon microdisks with BRAF siRNA-loaded liposomes (MNC/Lip-BRAF, 0.6 mg/kg BRAF siRNA) had the same efficacy as Taxotere® (Sanofi-Aventis, Bridgewater, N.J., USA) (4 mg/kg), inhibiting 40% and 38% of the nodule growth, respectively. The docetaxel-loaded PLGA-PEG nanoparticles (NP-Doc) with the inhibition rate of 58% presented better efficacy than Taxotere®, but they showed no significant difference with docetaxel-loaded PLGA-PEG nanoparticles plus BRAF siRNA-loaded liposomes (Lip-BRAF&NP-Doc), which also inhibited 58% of the nodule growth. The reason might be the rapid clearance of the siRNA-loaded liposomes.

When the PLGA-NPs were conjugated to the silicon microdisks (MNC/NP-Doc), it showed further improvement on inhibition of tumor growth than the NP-Doc group with the inhibition rate of 73%. Finally, with the same siRNA and docetaxel dose, the composite with both BRAF siRNA and docetaxel (MNC/Lip-BRAF and NP-Doc group) showed the best efficacy and almost totally inhibited the tumor growth in lungs with the inhibition rate of 89%, which was 1.2-fold more efficient than MNC/NP-Doc, 1.5-fold more efficient than Lip-BRAF&NP-Doc, 2.3-fold more efficient than Taxotereg.

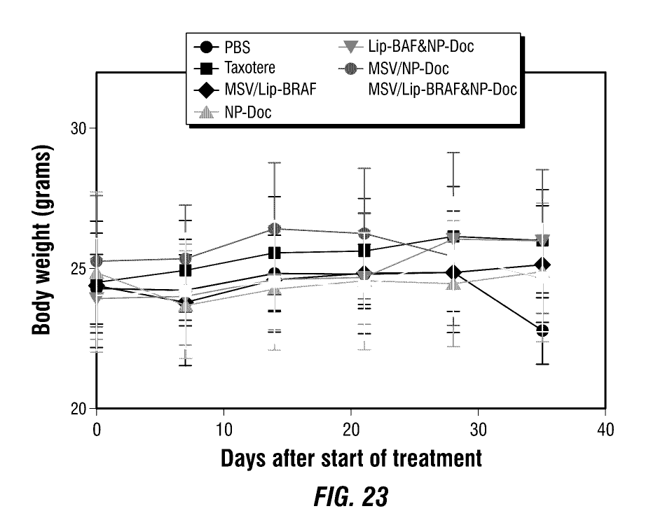

It is noteworthy that the mixture of Lip-BRAF and NP-Doc did not improve the efficacy of NP-Doc. However, once loading on the silicon microdisks, the MNC/Lip-BRAF and NP-Doc presented remarkable improvement than the MNC/NP-Doc, indicating that the composite could be an efficient platform for combination therapy in vivo. During the period of treatment (i.e., injection of the composition from Day 0, to Day 35), the body weight of the mice did not change significantly, except for the PBS group after 35 days, due to the serious lung metastasis (FIG. 23).

The survival curve also confirmed the efficacy of the composite (FIG. 19B). The first mouse died from the 39^{th} day after treatment in the PBS-negative group. After 81 days of treatment, 87.5% mice in MNC/Lip-BRAF and NP-Doc group were alive, while in the MNC/NP-Doc group, the Lip-BRAF and NP-Doc group, and the NP-Doc group, the survival rates were 50%, 25%, and 25%, respectively. All mice in the other groups died.

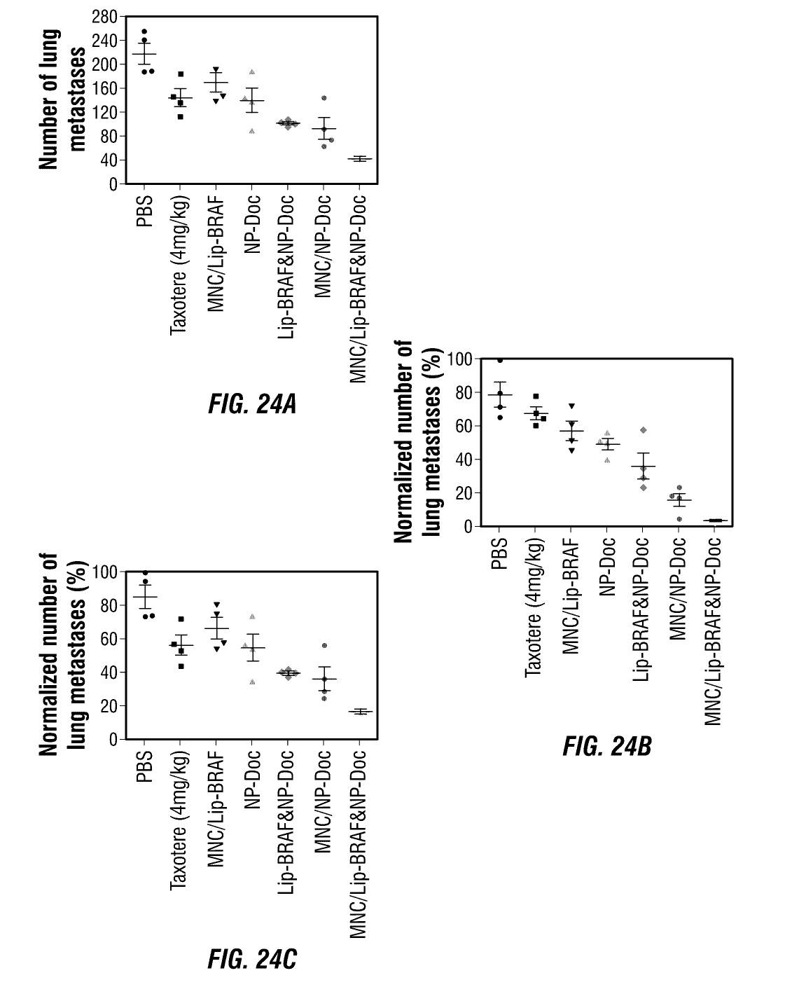

The mice in all groups were sacrificed at 35^{th} day after treatment and the lungs were collected for further analysis. The nodule numbers were recorded and they were consistent with the results from tumor growth curve and survival curve (FIG. 19C). In FIG. 19D, it showed the photos of lungs after treatment with different particles and lungs with different numbers of nodules could be observed directly. The histologic images with hematoxylin and eosin staining showed that after treatment with the composite, apparent disappearance of the nodules was observed and normal lung tissues were detected with no obvious pathological abnormalities (FIG. 19E, FIG. 24A, FIG. 24B, and FIG. 24C).

Mice with same stage of lung metastasis were used for immunohistochemistry (IHC) staining to show the decreased expression of BRAF in the tumor section after treatment with the composite (FIG. 19F). After 72 hrs' treatment with the particles, lung tissues were collected for IHC staining. Compared with PBS group, tumors treated with the MNC/Lip-BRAF and MNC/Lip-BRAF&NP-Doc exhibited a noticeable decrease in BRAF expression, which indicated effective delivery of the siRNA and therapeutic by the composite.

### CONCLUSION

A silicon-based micro/nano composite has been developed that permits simultaneous delivery of chemotherapeutic agents and siRNA to lung tissue. The composite was loaded with BRAF siRNA and docetaxel, and therapeutic efficacy was evaluated in A375 melanoma cells and in a mouse model of melanoma lung metastasis. The results revealed that a superior synergistic anti-cancer efficacy could be obtained both in vitro and in vivo using the delivery system of the present invention. In particular, treatment with the composite reduced the tumor burden, decreased the number of metastatic nodules, and dramatically prolonged survival in comparison to monotherapy or combination therapy with liposomes and polymers.

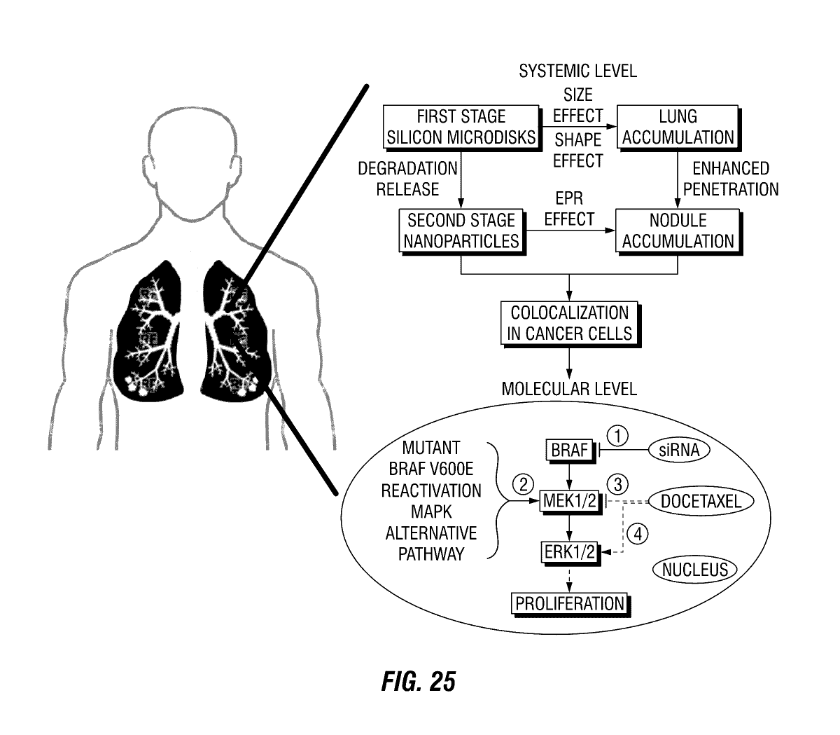

FIG. 25 depicts a schematic for the proposed mechanism of synergy. On the molecular level, several studies have demonstrated that cancer cells can acquire resistance to BRAF inhibitors (Nazarian et al., 2010; Johannessen et al., 2010; Poulikakos et al., 2011), primarily through reactivation of MEK (Das Thakur and Stuart, 2014). Here, docetaxel was shown to inhibit the MEK pathway, potentially sensitizing cells to BRAF inhibition. Furthermore, it has been shown that BRAF siRNA reversed docetaxel-induced overexpression of ERK, which has previously been linked to cancer cell survival (Mhaidat et al., 2007a; 2007b). Consequently, the combination of these therapeutic agents can act in a synergistic manner to prevent cancer cells from acquiring resistance to the therapy.

Moreover, therapeutic synergy is also highly dependent on coordinated spatiotemporal delivery of therapeutic agents. The results presented here demonstrate that micro/nano composite drug delivery systems were capable of simultaneously delivering an siRNA and a chemotherapeutic agent to metastatic melanoma lesions in mammalian lungs. Indeed, the microparticle component is designed to preferentially accumulate in lung vasculature following intravenous injection. Once the composite is lodged in pulmonary vessels, the nanoparticle component can infiltrate cancerous lesions by exploiting the EPR effect. On the contrary, when polymeric nanoparticles and liposomes were freely injected, the former caused accumulation in the liver and spleen, while the latter resulted in siRNA retention in the kidneys. Consequently, treatment with separate delivery vehicles failed to achieve synergistic anticancer efficacy, highlighting the importance of co-localized delivery of combination therapy.

Taken together, the composite is an efficient platform for treating melanoma lung metastasis, since it enables synergistic antitumor activity both on the molecular and systematic level. In light of these results, it is likely that the micro/nano composite can also be exploited to treat a variety of other pulmonary conditions. Because the improved efficacy of the micro/nano composite is mainly achieved by the enhanced accumulation and combination therapy, it can be also used for lung cancer (small cell lung cancer or non-small lung cancer) or other cancers lung metastasis (Breast cancer lung metastasis) through changing the suitable therapeutics).

Additionally, minor modifications to the composite could enable delivery of various therapeutic agents, thereby providing endless opportunities for new forms of combination therapy to the lungs such as other drugs like paclitaxel, inhibitors like dabrafenib or trametinib, or other siRNA like MEK siRNA or ERK siRNA.

### REFERENCES

The following references, to the extent that they provide exemplary procedural or other details supplementary to those set forth herein, are specifically incorporated herein in their entirety by express reference thereto:

ALEXIS, F et al., “Factors affecting the clearance and biodistribution of polymeric nanoparticles,” Mol. Pharm., 5(4):505-515 (July-August 2008).

ALMEIDA, J P et al., “In vivo biodistribution of nanoparticles,” Nanomedicine (London), 6(5):815-835 (July 2011).

BLANCO, E et al., “Colocalized delivery of rapamycin and paclitaxel to tumors enhances synergistic targeting of the PI3K/Akt/mTOR pathway,” Mol. Ther., 22(7):1310-1319 (July 2014).

CHENG, J et al., “Formulation of functionalized PLGA-PEG nanoparticles for in vivo targeted drug delivery,” Biomaterials, 28(5):869-876 (February 2007).

CHIAPPINI, C et al., “Tailored porous silicon microparticles: fabrication and properties,” ChemPhysChem., 11(5):1029-1035 (April 2010).

COFFEE, E M et al., “Concomitant BRAF and PI3K/mTOR blockade is required for effective treatment of BRAF(V600E) colorectal cancer,” Clin. Cancer Res., 19(10):2688-2698 (May 2013).

CRAFT, N et al., “Bioluminescent imaging of melanoma in live mice,” J. Invest. Dermatol., 125(1):159-165 (July 2005).

DAS THAKUR, M and STUART, D D, “Molecular pathways: response and resistance to BRAF and MEK inhibitors in BRAF(V600E) tumors,” Clin. Cancer Res., 20(5):1074-1080 (March 2014).

DAVIES, H et al., “Mutations of the BRAF gene in human cancer,” Nature, 417(6892):949-954 (June 2002).

DECUZZI, P et al., “Size and shape effects in the biodistribution of intravascularly injected particles,” J. Control Release, 141(3):320-327 (February 2010).

DORRESTEIJN, R et al. “Polylactide-block-polypeptide-block-polylactide copolymer nanoparticles with tunable cleavage and controlled drug release,” Adv. Funct. Mat., 24(26):4026-4033 (July 2014).

FENG, S S and CHIEN, S, “Chemotherapeutic engineering: Application and further development of chemical engineering principles for chemotherapy of cancer and other diseases,” Chem. Eng. Sci., 58(18):4087-4114 (September 2003).

FERRARI, M, “Cancer nanotechnology: opportunities and challenges,” Nat. Rev. Cancer, 5(3):161-171 (March 2005).

FERRARI, M, “Frontiers in cancer nanomedicine: directing mass transport through biological barriers,” Trends Biotechnol., 28(4):181-188 (April 2010).

FINE, D et al., “Silicon micro- and nanofabrication for medicine,” Adv. Healthc Mater., 2(5):632-666 (May 2013).

FLAHERTY, K T et al., “Improved survival with MEK inhibition in BRAF-mutated melanoma,” N. Engl. J. Med., 367(2):107-114 (July 2012).

FLAHERTY, K T et al., “Inhibition of mutated, activated BRAF in metastatic melanoma,” N. Engl. J. Med., 363(9):809-819 (August 2010).

FONSECA, C et al., “Paclitaxel-loaded PLGA nanoparticles: preparation, physicochemical characterization and in vitro anti-tumoral activity,” J. Controlled Rel., 83(2):273-286 (October 2002).

GARBER, K, “Melanoma combination therapies ward off tumor resistance,” Nat. Biotechnol., 31(8):666-668 (August 2013).

GEYSEN, H M et al., “Use of peptide synthesis to probe viral antigens for epitopes to a resolution of a single amino acid,” Proc. Nat'l. Acad. Sci. USA, 81:3998-4002 (July 1984).

GODIN, B et al., “Multistage nanovectors: from concept to novel imaging contrast agents and therapeutics,” Acc. Chem. Res., 44(10):979-989 (October 2011).

GRIBSKOV, M, and BURGESS, R R, “Sigma factors from E. coli, B. subtilis, phage SP01, and phage T4 are homologous proteins,” Nucleic Acids Res., 14(16):6745-6763 (1986).

GUPTA, A et al., “DOC-MEK: a double-blind randomized phase II trial of docetaxel with or without selumetinib in wild-type BRAF advanced melanoma,” Ann. Oncol., 25(5):968-974 (May 2014).

HALE, W G, and MARGHAM, J P, “HARPER COLLINS DICTIONARY OF BIOLOGY,” HarperPerennial, New York (1991).

HARDMAN, J G, and LIMBIRD, L E, (Eds.), “GOODMAN AND GILMAN'S THE PHARMACOLOGICAL BASIS OF THERAPEUTICS” 10^{th} Edition, McGraw-Hill, New York (2001).

HINGORANI, S R et al., “Suppression of BRAF(V599E) in human melanoma abrogates transformation,” Cancer Res., 63(17):5198-5202 (2003).

HUANG, X et al., “The shape effect of mesoporous silica nanoparticles on biodistribution, clearance, and biocompatibility in vivo,” ACS Nano., 5(7):5390-5399 (July 2011).

JANG, S and ATKINS, M B, “Which drug, and when, for patients with BRAF-mutant melanoma?” Lancet Oncol., 14(2):e60-69 (February 2013).

JOHANNESSEN, C M et al., “COT drives resistance to RAF inhibition through MAP kinase pathway reactivation,” Nature, 468(7326):968-972 (December 2010).

KAMALY, N et al., “Development and in vivo efficacy of targeted polymeric inflammation-resolving nanoparticles,” Proc. Nat'l. Acad. Sci. USA, 110(16):6506-6511 (April 2013).

KEARNEY, C J and MOONEY, D J, “Macroscale delivery systems for molecular and cellular payloads,” Nat. Mater., 12:1004-1017 (October 2013).

KIM, B Y S et al., “Nanomedicine,” N. Engl. J. Med., 363:2434-2443 (December 2010).

KUMAR, R et al., “BRAF mutations in metastatic melanoma: a possible association with clinical outcome,” Clin. Cancer Res., 9(9):3362-3368 (August 2003).

LI, L et al., “Correlation of growth capacity of human tumor cells in hard agarose with their in vivo proliferative capacity at specific metastatic sites,” J. Nat'l. Cancer Inst., 81(18):1406-1412 (September 1989).

MCARTHUR, G A et al., “Safety and efficacy of vemurafenib in BRAF(V600E) and BRAF(V600K) mutation-positive melanoma (BRIM-3): extended follow-up of a phase 3, randomised, open-label study,” Lancet Oncol., 15(3):323-332 (March 2014).

MHAIDAT, N M et al., “Docetaxel-induced apoptosis of human melanoma is mediated by activation of c-Jun NH₂-terminal kinase and inhibited by the mitogen-activated protein kinase extracellular signal-regulated kinase 1/2 pathway,” Clin. Cancer Res., 13(4):1308-1314 (February 2007).

MHAIDAT, N M et al., “Regulation of docetaxel-induced apoptosis of human melanoma cells by different isoforms of protein kinase C,” Mol. Cancer Res., 5(10):1073-1081 (October 2007).

NAZARIAN, R et al., “Melanomas acquire resistance to B-RAF(V600E) inhibition by RTK or N-RAS upregulation,” Nature, 468(7326):973-977 (November 2010).

NEEDLEMAN, S B and WUNSCH, C D, “A general method applicable to the search for similarities in the amino acid sequence of two proteins,” J. Mol. Biol., 48(3):443-453 (March 1970).

PEER, D et al., “Nanocarriers as an emerging platform for cancer therapy,” Nat. Nanotechnol., 2(12):751-760 (December 2007).

POULIKAKOS, P I et al., “RAF inhibitor resistance is mediated by dimerization of aberrantly spliced BRAF(V600E),” Nature, 480(7377):387-390 (November 2011).

RIBAS, A and FLAHERTY, K T, “BRAF targeted therapy changes the treatment paradigm in melanoma,” Nat. Rev. Clin. Oncol., 8(7):426-433 (May 2011).

RIEHEMANN, K et al., “Nanomedicine—challenge and perspectives,” Angew Chem. Int. Ed. Engl., 48(5):872-897 (January 2009).

SALAMA, A K and FLAHERTY, K T, “BRAF in melanoma: current strategies and future directions,” Clin. Cancer Res., 19(16):4326-4334 (August 2013).

SANHAI, W R et al., “Seven challenges for nanomedicine,” Nat. Nanotechnol., 3(5):242-244 (May 2008).

SEMETE, B et al., “In vivo evaluation of the biodistribution and safety of PLGA nanoparticles as drug delivery systems,” Nanomedicine (UK), 6(5):662-671 (October 2010).

SHANNON, A M et al., “The mitogen-activated protein/extracellular signal-regulated kinase kinase 1/2 inhibitor AZD6244 (ARRY-142886) enhances the radiation responsiveness of lung and colorectal tumor xenografts,” Clin. Cancer Res., 15(21):6619-6629 (November 2009).

SINGLETON, P and SAINSBURY, D, “DICTIONARY OF MICROBIOLOGY AND MOLECULAR BIOLOGY,” 2^{nd} Ed., John Wiley and Sons, New York (1987).

SMALLEY, K S and FLAHERTY, K T, “Integrating BRAF/MEK inhibitors into combination therapy for melanoma,” Br. J. Cancer, 100(3):431-435 (February 2009).

SUMIMOTO, H et al., “The BRAF-MAPK signaling pathway is essential for cancer-immune evasion in human melanoma cells,” J Exp. Med., 203(7):1651-1656 (June 2006).

TANAKA, T et al., “Sustained small interfering RNA delivery by mesoporous silicon particles,” Cancer Res., 70(9):3687-3696 (May 2010).

TASCIOTTI, E et al., “Mesoporous silicon particles as a multistage delivery system for imaging and therapeutic applications,” Nat. Nanotechnol., 3(3): 151-157 (March 2008).

TRAN, M A et al., “Targeting V600EB-Raf and Akt3 using nanoliposomal-small interfering RNA inhibits cutaneous melanocytic lesion development,” Cancer Res., 68(18):7638-7649 (September 2008).

VAN DE VEN, A L et al., “Rapid tumoritropic accumulation of systemically injected plateloid particles and their biodistribution,” J. Control Release, 158(1):148-155 (February 2012).

WELLBROCK, C, and MARAIS, R, “Elevated expression of MITF counteracts B-RAF-stimulated melanocyte and melanoma cell proliferation,” J. Cell Biol., 170(5): 703-708 (August 2005).

XU, L et al., “Gene expression changes in an animal melanoma model correlate with aggressiveness of human melanoma metastases,” Mol. Cancer Res., 6(5):760-769 (May 2008).

XU, R et al., “Multistage vectored siRNA targeting ataxia-telangiectasia mutated for breast cancer therapy,” Small, 9(9-10):1799-1808 (May 2013).

It should be understood that the examples and embodiments described herein are for illustrative purposes only and that various modifications or changes in light thereof will be suggested to persons skilled in the art and are to be included within the spirit and purview of this application and the scope of the appended claims.

All references, including publications, patent applications and patents, cited herein are hereby incorporated by reference to the same extent as if each reference was individually and specifically indicated to be incorporated by reference and was set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including,” or “containing,” with reference to an element or elements is intended to provide support for a similar aspect or embodiment of the invention that “consists of,” “consists essentially of,” or “substantially comprises,” that particular element or elements, unless otherwise stated or clearly contradicted by context (e.g., a composition described herein as comprising a particular element should be understood as also describing a composition that contains and/or that includes that particular element, unless otherwise explicated stated, or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically- and/or physiologically-related may be substituted for the agents described herein while the same or similar results would be achieved. All such similar substitutes and modifications apparent to those skilled in the art are deemed to be within the spirit, scope, and concept of the invention as defined by the appended claims.

What is claimed is:

1. A drug delivery system comprising:
(a) a micro/nano composite that comprises:
(i) a population of nanoparticles comprising a first therapeutic agent; and
(ii) a plurality of mesoporous microparticles,
wherein at least a first portion of the population of nanoparticles are sized and dimensioned to be contained within one or more pores of one or more of the plurality of mesoporous microparticles; and
(b) a buffer, diluent, or excipient.

2. The drug delivery system of claim 1, wherein the population of nanoparticles comprises one or more liposomes, one or more lipids, one or more lipid particles, one or more polymers, one or more micelles, one or more noble metals, one or more metal oxides, or one or more combinations thereof.

3. The drug delivery system of claim 2, wherein the one or more liposomes comprise 1,2-dioleoyl-sn-glycero-3-phosphocholine (DOPC), 1,2-dipalmitoylsn-glycero-3-phosphatidylcholine (DPPC), N-[1-(2,3-dioleoyloxy)propyl]-N,N,N-trimethylammonium chloride (DOTMA), N-[1-(2,3-dioleoyloxy)propyl]-N,N,N-trimethylammonium methyl sulfate (DOTAP), 1,2-dioleoyl-3-dimethylammonium-propane (DODAP), dimethyldioctadecylammonium bromide (DDAB), 3β-[N-(N′,N′-dimethylaminoethane)-carbamoyl]cholesterol hydrochloride (DCChol), 1,2-dioleoyl-sn-glycero-3-phosphoethanolamine (DOPE), dipalmitoylphosphatidylethanolamine (DPPE), or any combination thereof.

4. The drug delivery system of claim 1, wherein the population of nanoparticles comprises poly(lactic-co-glycolic) acid (PLGA), poly(L-lactide-co-glycolide) (PLLGA), polylactic acid (PLA), polyglycolic acid (PGA), polyethylene glycol (PEG), poly(L-Lactic-co-caprolactone) (PLCL), poly(caprolactone) (PCL), polyurethane (PU), or any combination thereof.

5. The drug delivery system of claim 4, wherein the population of nanoparticles comprises PLGA and PEG.

6. The drug delivery system of claim 1, wherein the nanoparticles are about 60 to about 500 nm in average diameter.

7. The drug delivery system of claim 6, wherein the nanoparticles are about 100 to about 300 nm in average diameter.

8. The drug delivery system of claim 1, wherein the plurality of mesoporous microparticles comprises mesoporous silicon microdisks.

9. The drug delivery system of claim 8, wherein the mesoporous silicon microdisks are substantially about 1 μm to about 10 μm in diameter.

10. The drug delivery system of claim 8, wherein the mesoporous silicon microdisks are substantially about 0.1 μm to about 5 μm in height.

11. The drug delivery system of claim 8, wherein the pores of the mesoporous silicon microdisks are substantially 0 to about 60 nm in diameter.

12. The drug delivery system of claim 11, wherein the pores of the mesoporous silicon microdisks are substantially about 10 to about 40 nm in diameter.

13. The drug delivery system of claim 1, wherein the plurality of mesoporous microparticles are adapted configured to release the first therapeutic agent from the population of nanoparticles: (a) in response to an external stimulus; (b) in response to a change in the environment of the micro/nano composite; or (c) as a result of degradation of at least a first portion of the plurality of mesoporous microparticles.

14. The drug delivery system of claim 13, wherein the degradation of the at least a first portion of the plurality of mesoporous microparticles occurs via enzyme-facilitated biodegradation of the microparticle substrate.

15. The drug delivery system of claim 1, further comprising a cellular-targeting moiety.

16. The drug delivery system of claim 15, wherein the cellular-targeting moiety is operably linked to a first outer surface of at least a first portion of the plurality of mesoporous microparticles.

17. The drug delivery system of claim 15, wherein the cellular-targeting moiety is selected from the group consisting of a chemically-targeting moiety, a physically-targeting moiety, a geometrically-targeting moiety, a ligand, a ligand-binding moiety, a receptor, a receptor-binding moiety, an antibody, and antigen-binding fragment, and any combination thereof.

18. The drug delivery system of claim 15, wherein the cellular-targeting moiety comprises a plurality of distinct antigenic ligands that elicit one or more target-specific immune responses in a mammalian host cell contacted with the micro/nano composite.

19. The drug delivery system of claim 1, further comprising a diagnostic reagent or a detectable label.

20. The drug delivery system of claim 19, wherein the diagnostic reagent or the detectable label comprises an imaging reagent, a contrast reagent, a fluorescent label, a radiolabel, a magnetic resonance imaging (MRI) label, a spin label, or any combination thereof.

21. The drug delivery system of claim 17, wherein the chemically-targeting moiety is disposed on, conjugated to, or chemically cross-linked to, a first portion of the outer surface of the plurality of mesoporous microparticles, and comprises a ligand, a dendrimer, an oligomer, an aptamer, a binding protein, an antibody, an antigen-binding fragment, a receptor, a targeting peptide, or any combination thereof.

22. The drug delivery system of claim 1, wherein the first therapeutic agent comprises a molecule selected from the group consisting of an immune-stimulating agent, a tumor growth inhibitor, a protein, a peptide, a small molecule, an RNA, a DNA, an siRNA, an aptamer, and an antibody.

23. The drug delivery system of claim 4, wherein the first therapeutic agent comprises a chemotherapeutic drug selected from the group consisting of axitinib, bevacizumab, binimetinib, carboplatin, cobimetinib, dabrafenib, dacarbazine, docetaxel, encorafenib, ipilumimab, oblimersen, paclitaxel, selumetinib, sorafenib, trametinib, vemurafenib, and combinations thereof.

24. The drug delivery system of claim 1, wherein the first therapeutic agent comprises a siRNA that is specific for a mammalian gene selected from the group consisting of BRAF, MEK, ERK1, and ERK2.

25. The drug delivery system of claim 1, adapted and configured as part of a therapeutic kit that comprises the micro/nano composite, and at least a first set of instructions for administration of the micro/nano composite to a mammal in need thereof.

26. A population of isolated mammalian cells comprising a micro/nano composite that (i) a population of nanoparticles comprising a first therapeutic agent; and (ii) a plurality of mesoporous microparticles, wherein at least a first portion of the population of nanoparticles are sized and dimensioned to be contained within one or more pores of one or more of the plurality of mesoporous microparticles, wherein at least a first portion of the population of nanoparticles are contained within one or more pores of one or more of the plurality of silicon microparticles.

27. A kit comprising the drug delivery system of claim 1, and instructions for administering the micro/nano composite to a mammal in need thereof, as part of a regimen for the prevention, diagnosis, treatment, or amelioration of one or more symptoms of a disease, a dysfunction, an abnormal condition, or a trauma in the mammal.

28. A method for providing one or more antigens to a population of cells within the body of an animal, comprising administering to the animal an amount of the drug delivery system of claim 1, for a time effective to provide the one or more antigens to the population of cells within the body of the animal.

29. The method of claim 28, wherein the animal is at risk for developing, is suspected of having, or is diagnosed with a tumor or a cancer.

30. The method of claim 29, wherein the animal has been diagnosed with lung cancer.

31. The method of claim 30, wherein the animal is a human that has been diagnosed with melanoma metastatic cancer of the lung.

32. A method of administering a diagnostic, therapeutic, or prophylactic agent to one or more cells, tissues, organs, or systems of a mammalian subject in need thereof, comprising administering to the subject an effective amount of the drug delivery system of claim 1.

33. The method of claim 32, wherein the diagnostic, therapeutic, or prophylactic agent comprises at least a first siRNA, a first chemotherapeutic agent, or a combination thereof.
